(19) United States
(12) Patent Application Publication
(10) Pub. No.: US 2021/0043284 A1
(43) Pub. Date: Feb. 11, 2021

(54) DENIABLE DIGITAL HEALTH DIAGNOSES

(71) Applicant: Healthblock, Inc., Palo Alto, CA (US)

(72) Inventors: Jan T. LIPHARDT, Palo Alto, CA (US); Brian JUN, Mountain View, CA (US)

(21) Appl. No.: 16/537,621

(22) Filed: Aug. 11, 2019

(51) Int. Cl. G16H 10/40; G16H 10/60; G06F 21/62

(52) U.S. Cl. CPC G16H 10/40; G16H 15/00; G06F 21/6254; G16H 10/60

(57) ABSTRACT

Methods, systems, and computer program products for health data management. A user obtains a biomaterial collection kit that provides a unique identifier that is matched to a biomaterial specimen container. The identity of any user is not associated with any unit of the biomaterial kit. The user retains their unique identifier and mails their biomaterial (e.g., hair, saliva, etc.) anonymously to a lab. The lab analyzes the biomaterial sample to produce anonymous analysis results. The lab broadly publishes the anonymous analysis results to a publicly-accessible network location (e.g., the Internet). At will, users access the publicly-accessible repository to initiate compute operations on the published entries. If a particular user's identifier matches a published entry (e.g., if the entry can be decrypted using that particular user's unique identifier), then that user can gain access to that published entry while being unable to match to or decrypt any other user's published entries.

### FIELD

This disclosure relates to health data management and more particularly to techniques for deniable digital health diagnoses.

### BACKGROUND

The emergence and adoption of modern technologies such as smart phones, social networks, and internet applications is changing not only how we communicate, but is also changing how we request, deliver, and receive health care. The use of such modern technologies in a health care ecosystem is often referred to as “digital health”. The expectation of the participants (e.g., patients, physicians, hospitals, pharmacies, pharmaceutical companies, etc.) in a digital health ecosystem is that these technologies will improve health care efficiencies and/or health care outcomes. As such, the proliferation of digital health initiatives and applications is growing at a rapid pace.

In today's digital health ecosystem, vast amounts of patient health information (e.g., diagnoses, treatment history, medications, etc.) are stored electronically in many disparate locations and frequently accessed and/or shared by large numbers of participants in the ecosystem. Various laws, regulations, guidelines, and other types of governance have been enacted to establish bounds for managing the disclosure and ongoing safekeeping of such health information while considering the privacy rights and/or preferences of the participants. In the United States, for example, the Security Rule of the Health Insurance Portability and Accountability Act (HIPAA) specifically addresses the handling of protected health information (PHI). Specifically, the Security Rule of HIPAA was established to protect a patient's personally identifiable information (PII) while still allowing digital health ecosystem participants access to needed PHI. The Security Rule of HIPAA supports flexible adoption of technologies that facilitate the handling of PHI. Whether by reasons of law or for other reasons, patients, health care providers, insurance companies, banks, and other entities that handle health information all have strong incentives to protect sensitive health information.

Unfortunately, while the disclosure and distribution of a patient's health information may be governed according to HIPAA and/or other laws, regulations, or incentives, there remain associations between a particular patient's health information and the corresponding patient. As an example, a patient who visits a doctor to obtain a diagnosis pertaining to some health condition is “known” by the doctor, and any diagnosis associated with the patient is documented in the patient's records. Even though the doctor might properly observe PHI governance (e.g., in strict observance of all applicable HIPAA rules, in strict observance of patient-doctor confidentiality, etc.), the association between the patient and the diagnoses nevertheless exists and is thus subject to intentional and/or accidental disclosure.

Furthermore, even when a patient uses digital technology to interface with a health care provider, a digital trace (e.g., electronic information comprising PII, IP addresses, user identifiers, device identifiers, etc.) of the interaction is often sufficient for determining an association between the patient and his or her health information (e.g., a diagnosis, etc.). As such, current approaches to managing the privacy of digital health information fail to dissociate patients from their health information and, as a consequence, a patient is unable to achieve anonymity as pertains to his or her health information. This lack of patient anonymity is often of great concern to patients that desire “deniable diagnoses.” Deniable diagnoses are dissociated from respective patients so as to allow any particular patient to “deny” that a diagnosis was ever requested, determined, and/or delivered. For example, a patient might want to be able to deny that a diagnosis had been made in situations when a particular diagnosis—or even a request for a diagnosis—can bring about a personal and/or family stigma, and/or a denial of services, and/or other negative consequences. What is needed is a way for health care providers to deliver deniable diagnoses to patients.

### SUMMARY

The present disclosure describes techniques used in systems, methods, and in computer program products for deniable digital health diagnoses, which techniques advance the relevant technologies to address technological issues with legacy approaches. Certain embodiments are directed to technological solutions for implementing an anonymous health data management framework to secretly match a particular anonymous user to his or her anonymous health data that is published to an Internet-accessible location for access only by the particular anonymous user.

The disclosed embodiments modify and improve over legacy approaches. In particular, the herein-disclosed techniques provide technical solutions that address the technical problems attendant to anonymously delivering diagnoses and/or other health information to anonymous patients in a digital health ecosystem. Some of such technical solutions involve specific implementations (i.e., data organization, data communication paths, module-to-module interrelationships, etc.) that relate to the software arts for improving computer functionality. For example, the disclosed anonymous data repository comprises a digest of entries that are configured to be efficiently scanned by a participant so as to reduce the latency and/or computing resource consumption when accessing anonymous health data of the anonymous data repository. More specifically, the data structures as disclosed herein and their use serve to reduce both memory usage and CPU cycles as compared to alternative approaches.

The ordered combination of steps of the embodiments serve in the context of practical applications that perform steps for implementing an anonymous health data management framework to secretly match a particular anonymous user to his or her anonymous health data that is published to a network-accessible repository for access only by the particular anonymous user. Specifically, the disclosed embodiments for implementing an anonymous health data management framework to secretly match a particular anonymous user to his or her anonymous health data involves technological solutions that pertain to technological problems that arise in the hardware and software arts that underlie digital health record ecosystems. Aspects of the present disclosure achieve performance and other improvements in peripheral technical fields including (but not limited to) anonymous digital publishing and cyber threat countermeasures.

This disclosure includes many embodiments of many practical applications. In addition to the disclosed methods specific to implementing an anonymous digital health data management framework, other practical applications pertain to availability and use of a biomaterials collection and delivery kit. Such a kit comprises a biomaterial container having an association to an identification code, and a user card having printed thereon the identification code. The biomaterial container is configured with an association to the identification code. The identification code does not include any personally identifiable information. Moreover, the identification code does not include any patient health information. The contents of the biomaterial container are used to generate anonymous analysis results, which anonymous analysis results are published to a data repository that comprises anonymous health data entries. The anonymous user, possibly using an app, is able to form an access request that comprises at least a portion of the identification code. The identification code or portion thereof is used for matching to at least one of the anonymous health data entries.

An additional practical application pertains to executable code on a user device (e.g., an app), which executable code implements portions of a method for delivering deniable digital health diagnoses. Such executable code is posted as a downloadable instance of a software application. An agent (e.g., a host of a download service) responds to a request to download the software application. The software application is configured to carry out steps of (1) initializing itself on a user device; and (2) accessing a health data entry provided by a health care provider, where the health data entry corresponds to a subject anonymous user.

The app is not notified when the health care provider provides anonymous analysis results in response to analyzing biomaterials from a plurality of anonymous users (e.g., including the user who downloads the app); however, the anonymous analysis results are published to a data repository as anonymous health data comprising entries that are published in response to provisions of the anonymous analysis results by the health care provider. Periodically, the app forms access requests to be processed over the data repository so as to match the subject anonymous user with at least one of the entries.

Further details of aspects, objectives, and advantages of the technological embodiments are described herein and in the drawings and claims.

### BRIEF DESCRIPTION OF THE DRAWINGS

The drawings described below are for illustration purposes only. The drawings are not intended to limit the scope of the present disclosure.

FIG. 1A and FIG. 1B illustrate a computing environment in which embodiments of the present disclosure can be implemented.

FIG. 2 depicts an anonymous health data delivery technique as implemented in systems that facilitate deniable digital health diagnoses, according to an embodiment.

FIG. 3A and FIG. 3B depict a system that implements deniable digital health diagnoses, according to an embodiment.

FIG. 3C illustrates a health data management framework that facilitates deniable digital health diagnoses, according to an embodiment.

FIG. 4A presents an anonymous biomaterials processing technique as implemented in systems that facilitate deniable digital health diagnoses, according to an embodiment.

FIG. 4B presents a biomaterials delivery kit preparation technique as implemented in systems that facilitate deniable digital health diagnoses, according to an embodiment.

FIG. 5 depicts an anonymous health data publication technique as implemented in systems that facilitate deniable digital health diagnoses, according to an embodiment.

FIG. 6 presents an anonymous health data access technique as implemented in systems that facilitate deniable digital health diagnoses, according to an embodiment.

FIG. 7 depicts an anonymous response publication technique as implemented in systems that facilitate deniable digital health diagnoses, according to an embodiment.

FIG. 8 presents an anonymous response access technique as implemented in systems that facilitate deniable digital health diagnoses, according to an embodiment.

FIG. 9A and FIG. 9B depict systems that implement certain of the herein-disclosed embodiments.

FIG. 10A and FIG. 10B present block diagrams of computer system architectures having components suitable for implementing embodiments of the present disclosure, and/or for use in the herein-described environments.

### DETAILED DESCRIPTION

Aspects of the present disclosure solve problems associated with using computer systems for delivering diagnoses and/or other health information to anonymous patients in a digital health ecosystem. These problems are unique to, and may have been created by, various computer-implemented methods for delivering diagnoses and/or other health information to anonymous patients in a digital health ecosystem in the context of digital health record ecosystems. Some embodiments are directed to approaches for implementing an anonymous health data management framework to secretly match a particular anonymous user to his or her anonymous health data that is published for access only by the particular anonymous user. The accompanying figures and discussions herein present example environments, systems, methods, and computer program products for handling deniable digital health diagnoses.

### Overview

Disclosed herein are techniques for implementing an anonymous health data management framework to secretly match anonymous users with respective sets of anonymous health data that are published for access only by particular anonymous users. In certain embodiments, the framework is implemented in a digital health ecosystem having a plurality of anonymous users (e.g., patients) that submit respective collections of anonymous biomaterials for analysis by a plurality of health care providers. The biomaterials received by the health care providers are “anonymous” since the biomaterials are dissociated from any particular one of the anonymous users. The results produced in response to receiving the anonymous biomaterials are published to an anonymous health data repository as respective anonymous health data entries. The anonymous users scan the anonymous health data repository to secretly discover the entry or entries that were published to the repository in response to the analysis being performed over their respective biomaterials. As an example, a subject anonymous user that had submitted certain subject anonymous biomaterial for analysis will be able to access only the subject anonymous health data entries that were published in response to performance of the analysis of the subject anonymous biomaterial. Moreover, the subject anonymous user will not be able to access any health data entries other than his or her own health data entries. Furthermore, any others of the anonymous users will not be able to access a particular subject's anonymous health data entries. As such, according to the herein disclosed techniques, an anonymous user can submit anonymous biomaterials and access anonymous health data with complete anonymity and while leaving only a negligible digital trace.

In certain embodiments, an anonymous user that has achieved access to certain anonymous health data entries can publish one or more anonymous responses to an anonymous response repository. The health care providers scan the anonymous response repository to secretly discover the response or responses that were published to the repository in response to respective biomaterial analyses performed by the health care providers. In certain embodiments, publication of and access to the anonymous health data entries and/or anonymous responses are facilitated by a key-based encryption scheme. In certain embodiments, the functions and/or operations of the anonymous health data management framework are facilitated by deployment of a user device application or “app” (e.g., a downloadable software application) that interfaces with a publication service, a content access service, and other computing entities.

### Definitions and Use of Figures

Some of the terms used in this description are defined below for easy reference. The presented terms and their respective definitions are not rigidly restricted to these definitions—a term may be further defined by the term's use within this disclosure. The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application and the appended claims, the term “or” is intended to mean an inclusive “or” rather than an exclusive “or”. That is, unless specified otherwise, or is clear from the context, “X employs A or B” is intended to mean any of the natural inclusive permutations. That is, if X employs A, X employs B, or X employs both A and B, then “X employs A or B” is satisfied under any of the foregoing instances. As used herein, at least one of A or B means at least one of A, or at least one of B, or at least one of both A and B. In other words, this phrase is disjunctive. The articles “a” and “an” as used in this application and the appended claims should generally be construed to mean “one or more” unless specified otherwise or is clear from the context to be directed to a singular form.

Various embodiments are described herein with reference to the figures. It should be noted that the figures are not necessarily drawn to scale, and that elements of similar structures or functions are sometimes represented by like reference characters throughout the figures. It should also be noted that the figures are only intended to facilitate the description of the disclosed embodiments—they are not representative of an exhaustive treatment of all possible embodiments, and they are not intended to impute any limitation as to the scope of the claims. In addition, an illustrated embodiment need not portray all aspects or advantages of usage in any particular environment.

An aspect or an advantage described in conjunction with a particular embodiment is not necessarily limited to that embodiment and can be practiced in any other embodiments even if not so illustrated. References throughout this specification to “some embodiments” or “other embodiments” refer to a particular feature, structure, material or characteristic described in connection with the embodiments as being included in at least one embodiment. Thus, the appearance of the phrases “in some embodiments” or “in other embodiments” in various places throughout this specification are not necessarily referring to the same embodiment or embodiments. The disclosed embodiments are not intended to be limiting of the claims.

### Descriptions of Example Embodiments

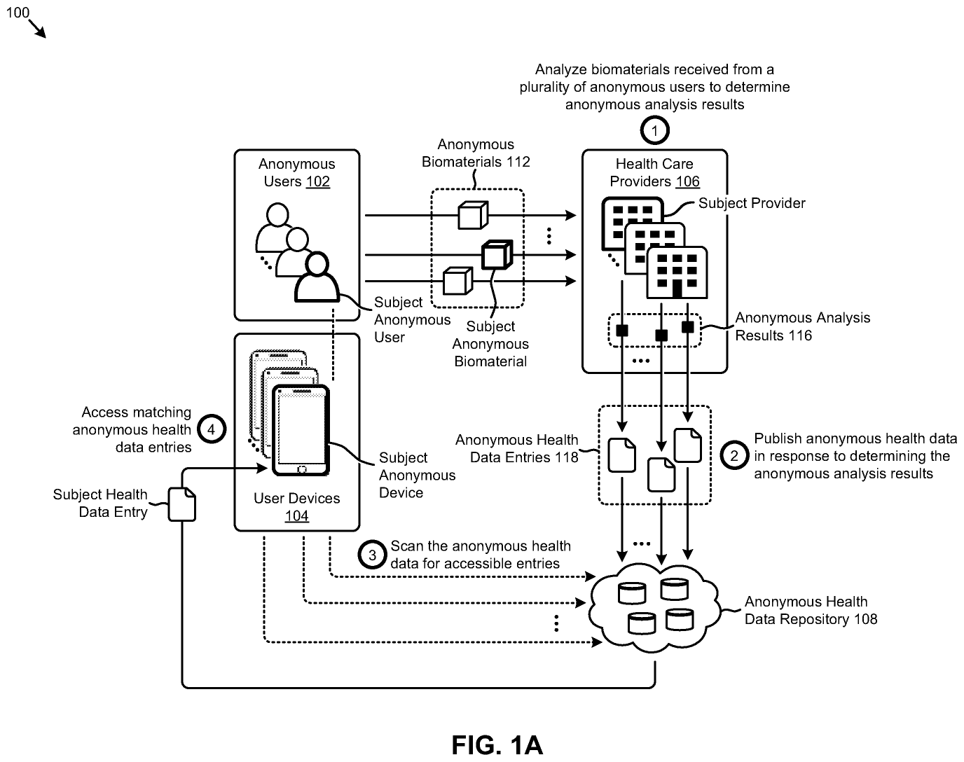

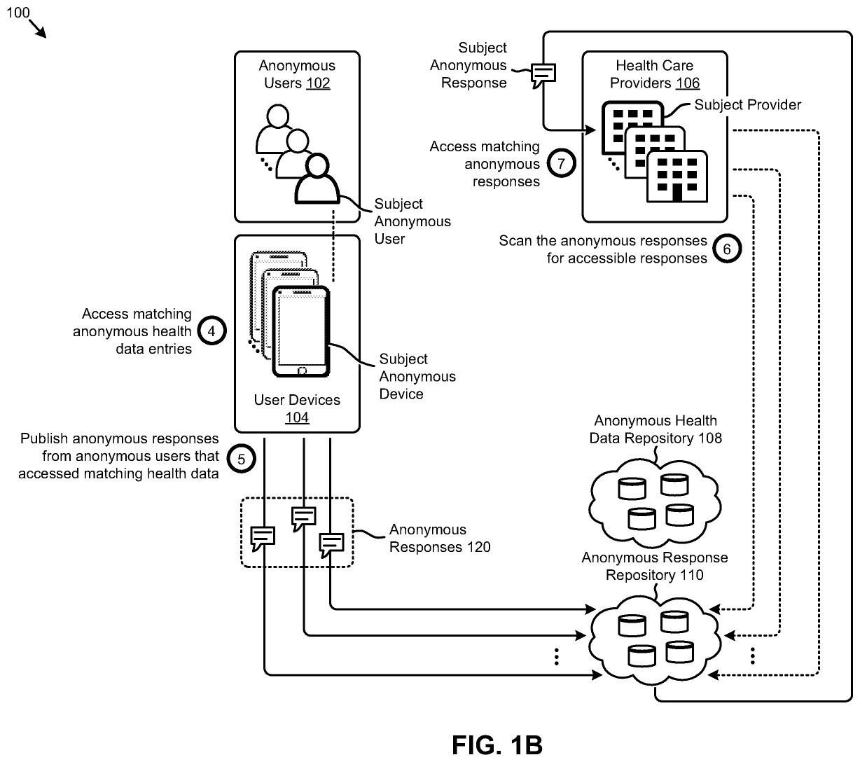

FIG. 1A and FIG. 1B illustrate a computing environment 100 in which embodiments of the present disclosure can be implemented. As an option, one or more variations of computing environment 100 or any aspect thereof may be implemented in the context of the architecture and functionality of the embodiments described herein.

FIG. 1A and FIG. 1B illustrate aspects pertaining to implementing an anonymous health data management framework to secretly match a particular anonymous user to his or her anonymous health data that is published for access only by the particular anonymous user. Specifically, the figure presents a logical depiction of how the herein disclosed techniques can be implemented to facilitate the delivery of user-specific analysis results, diagnoses, and/or other health information to users in a digital health ecosystem while maintaining the anonymity of the users. A representative set of high order operations are also presented to illustrate how the herein disclosed techniques might be applied in computing environment 100.

The logical depiction of FIG. 1A illustrates a computing environment comprising a plurality of anonymous users 102 (e.g., patients) who desire to receive user-specific health information from a plurality of health care providers 106 in a digital health ecosystem. As used herein, an anonymous user is a user (e.g., patient) that achieves anonymity from any participant in the digital health ecosystem that accesses or otherwise handles user-specific health information corresponding to the user. For example, a user may desire to be an anonymous user to a health care provider who provides a user-specific diagnosis corresponding to the user so that the user can deny that the diagnosis was ever requested, determined, and/or delivered. In the scenario of FIG. 1A, anonymous users 102 desire to obtain analysis results from health care providers 106 that are derived from respective sets of anonymous biomaterials 112 delivered from the anonymous users to the health care providers. As earlier mentioned however, current approaches to managing digital health information fail to dissociate users from their health information and, as a consequence, a user is unable to achieve anonymity from other participants in a digital health ecosystem.

The herein disclosed techniques address such deficiencies attendant to maintaining user anonymity by secretly matching anonymous users with respective sets of anonymous health data that are published for access only by particular anonymous users. As used herein, anonymous health data comprises health data entries that are dissociated from any one particular user. As such, the health data entries can be referred to as anonymous health data entries. For example, an anonymous health data entry that is included in a set of anonymous health data might describe certain analysis results (e.g., diagnoses) that pertain to a particular user, but with no evidence (e.g., digital trace, PII, etc.) of an association with the user being codified into or otherwise present in the anonymous health data entry. In some cases, to further protect the anonymity of the user, the analysis results described by the anonymous health data entry may be anonymous analysis results, which are analysis results that pertain to a particular user, but that have no evidence of an association with the user in the analysis results.

As can be observed in FIG. 1A, and according to the herein disclosed techniques, anonymous biomaterials 112 received from anonymous users 102 are analyzed by health care providers 106 to determine respective sets of anonymous analysis results 116 (operation 1). Anonymous biomaterials 112 often comprise biomaterial samples (e.g., saliva sample, stool sample, hair sample, small blood sample, etc.) that can be extracted by the users with no involvement from other participants. In some cases, other participants (e.g., phlebotomists, doctors, etc.) may be involved in extracting the biomaterial samples (e.g., large blood sample, etc.) from the users. In such cases, the biomaterials delivered to health care providers 106 can be anonymous biomaterials 112 if care is taken to dissociate the anonymous users 102 from the anonymous biomaterials 112. For example, a genome sequencing provider might receive a blood sample extracted from an anonymous user by a phlebotomist and generate anonymous analysis results that comprise a genome sequence derived from the blood sample.

In response to determining the anonymous analysis results, sets of anonymous health data are published to a data repository (operation 2). More specifically, in response to anonymous analysis results 116 being determined by health care providers 106, respective instances of anonymous health data entries 118 comprising the results are published to an anonymous health data repository 108. Anonymous users 102 interact with their respective user devices 104 to scan the anonymous health data repository 108 to secretly discover the instance or instances of anonymous health data entries 118 that were published to the repository in response to the analysis performed over their respective anonymous biomaterials (operation 3). When matches between anonymous users 102 and anonymous health data entries 118 are discovered, the matching entries are accessed at the respective user devices (operation 4). In accordance with the herein disclosed techniques, the foregoing scanning, matching, and accessing is performed with a negligible digital trace to protect the anonymity of anonymous users 102.

As illustrated in the example depicted in FIG. 1A, a subject anonymous user that had submitted certain subject anonymous biomaterials for analysis by a subject provider will be able to access, using a subject anonymous device, only a subject anonymous health data entry that was published in response to determining certain anonymous analysis results from the subject anonymous biomaterial. Moreover, the subject anonymous user will not be able to access any of the anonymous health data entries 118 in anonymous health data repository 108 that are derived from analyses of anonymous biomaterials other than the subject anonymous biomaterial. Furthermore, any of the other anonymous users 102 will not be able to access the subject anonymous health data entry in anonymous health data repository 108. The subject provider may publish an update to the subject anonymous health data entry but cannot otherwise access the subject anonymous health data entry or any other anonymous health data entries 118 in anonymous health data repository 108. As such, according to the herein disclosed techniques, anonymous users 102 can submit the anonymous biomaterials 112 and access the anonymous health data entries 118 with complete anonymity and a negligible digital trace.

Referring to FIG. 1B, any of the anonymous users 102 that have achieved access to respective instances of anonymous health data entries from anonymous health data repository 108 (operation 4) can publish one or more instances of anonymous responses 120 to an anonymous response repository 110 (operation 5). As an example, the subject anonymous user who successfully accesses the subject health data entry at the subject anonymous device might issue a subject anonymous response from the subject anonymous device to anonymous response repository 110. As used herein, an anonymous response is an electronic message that is dissociated from any particular user.

As an example, an anonymous response might comprise a question pertaining to one or more results received by a user. Health care providers 106 scan the anonymous response repository 110 to secretly discover the instance or instances of anonymous responses 120 that are accessible by the respective providers (operation 6). For example, the subject provider might scan the anonymous response repository 110 to discover the subject anonymous response issued by the subject anonymous user. When matches between health care providers 106 and anonymous responses 120 are discovered, the matching responses are accessed by the health care providers (operation 7). In accordance with the herein disclosed techniques, anonymity of the users issuing the responses is achieved with the health care providers receiving the responses, and with other participants in the health care ecosystem.

One embodiment of techniques for facilitating delivery of anonymous health data to anonymous participants in a digital health ecosystem is disclosed in further detail as follows.

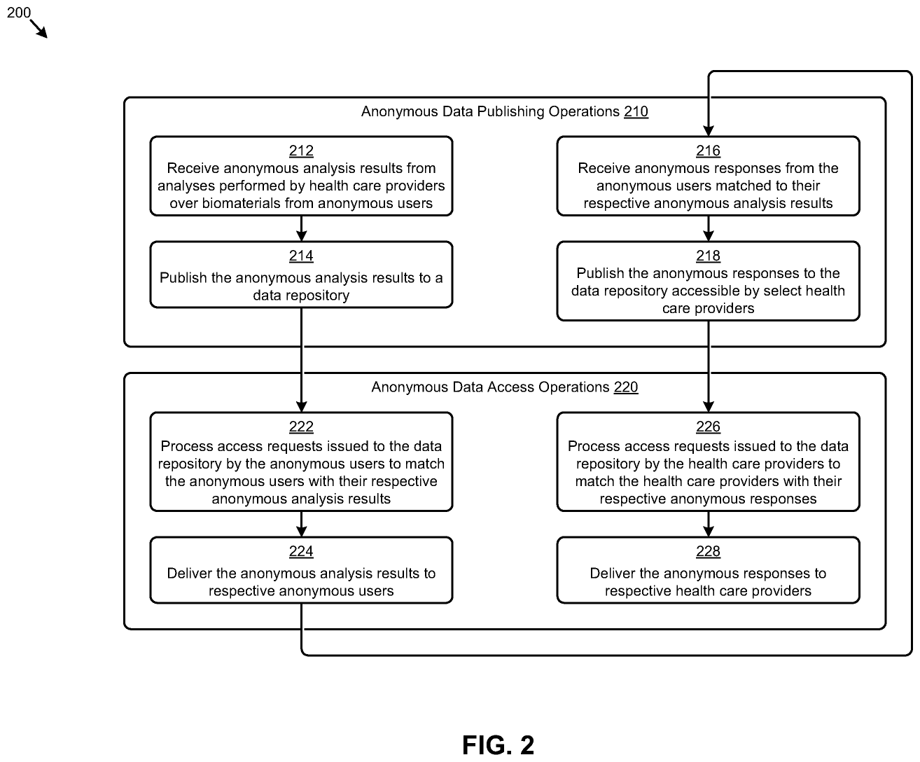

FIG. 2 depicts an anonymous health data delivery technique 200 as implemented in systems that facilitate deniable digital health diagnoses. As an option, one or more variations of anonymous health data delivery technique 200 or any aspect thereof may be implemented in the context of the architecture and functionality of the embodiments described herein. The anonymous health data delivery technique 200 or any aspect thereof may be implemented in any environment.

FIG. 2 illustrates aspects pertaining to implementing an anonymous health data management framework to secretly match a particular anonymous user to his or her anonymous health data that is published for access only by the particular anonymous user. Specifically, the figure is presented to illustrate one embodiment of certain steps and/or operations performed over a network of devices (e.g., user devices, servers, computing systems, etc.) to deliver anonymous health data to respective anonymous participants (e.g., users, patients, health care providers, etc.) in a digital health ecosystem. As can be observed, the steps and/or operations can be grouped into a set of anonymous data publishing operations 210 and a set of anonymous data access operations 220.

The anonymous data publishing operations 210 of anonymous health data delivery technique 200 commences by receiving anonymous analysis results generated from analyses performed by health care providers over biomaterials submitted by a plurality of anonymous users (step 212). In an exemplary case, the biomaterials are anonymous biomaterials in that no participant in the ecosystem other than the anonymous users from which the biomaterials are extracted has knowledge of any association between the biomaterials and the anonymous users. In certain cases, a medical practitioner may be needed to extract biomaterials from a particular user. In such cases, the anonymity of the biomaterials and users can be achieved without the health care providers and/or any other downstream participants in the digital health ecosystem having knowledge of any user's identity. The anonymous analysis results received from the health care providers are published to a data repository (step 214). As described herein, the anonymous analysis results are often codified into some form of an anonymous health data entry, such as a health report and/or data file and/or data folder, that is then published to the repository.

According to the anonymous data access operations 220, access requests issued to the data repository by the anonymous users are processed to match the anonymous users with their respective anonymous analysis results (step 222). In this case, a subject anonymous user will be matched with a respective set of subject anonymous analysis results (e.g., a file comprising the results) that was published to the data repository in response to analyzing the anonymous biomaterials from the subject anonymous user. The subject anonymous user will not be matched with any other anonymous analysis results, nor will any other anonymous users be matched with the subject user's anonymous analysis results. In response to discovering one or more matches, the anonymous analysis results are delivered for access by the respective anonymous users (step 224). As an example, the anonymous users might review their respective anonymous results on their personal user device (e.g., smart phone, laptop computer, etc.).

In certain embodiments, the roles of anonymous data publisher and the anonymous data requestor in the digital health ecosystem can be reversed or otherwise accepted by various participants in the ecosystem. As indicated in the anonymous data publishing operations 210 of anonymous health data delivery technique 200, anonymous responses received from anonymous users that match to their respective anonymous analysis results might be received (step 216) and published to the data repository for access by certain health care providers (step 218). As an example, a subject anonymous response might comprise a question and/or comment and/or additional follow-up information pertaining to the subject anonymous analysis results received by the subject anonymous user.

According to the anonymous data access operations 220, access requests issued to the data repository by the health care providers are processed to match the health care providers with their respective anonymous responses (step 226). In this case, the subject provider who published the subject anonymous analysis results will be matched with the subject anonymous response from the subject anonymous user. The subject provider will not be matched with any other anonymous responses, nor will any other health care providers be matched with the subject anonymous response. When matching anonymous responses are discovered, they are delivered for access by the respective health care providers (step 228).

One embodiment of a system, data flows, and data structures for implementing the anonymous health data delivery technique 200 and/or other herein disclosed techniques, is disclosed as follows.

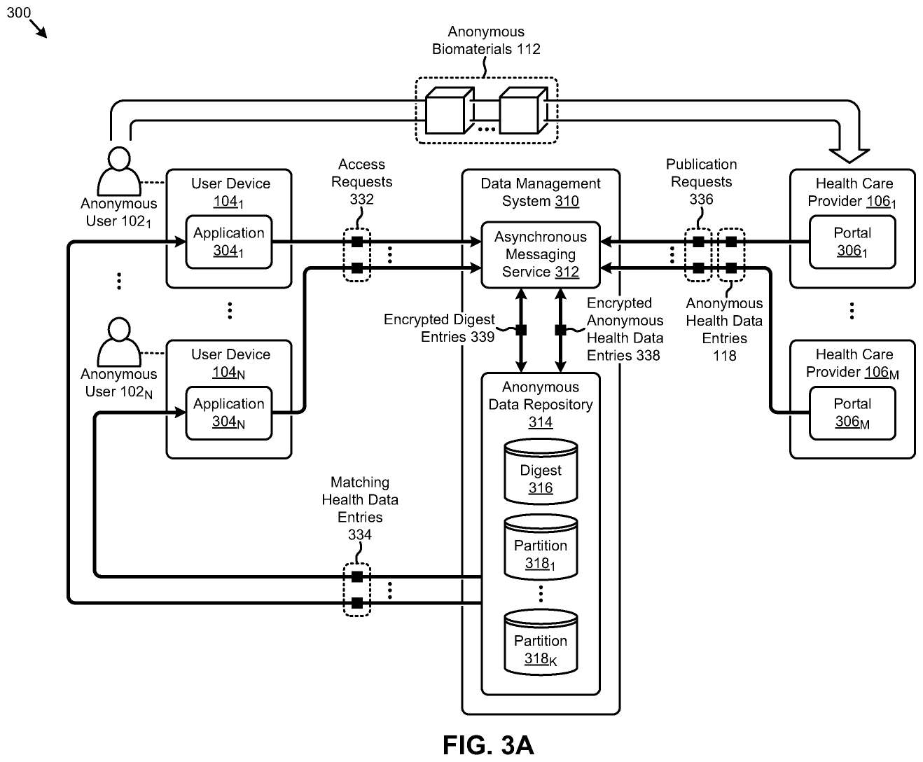

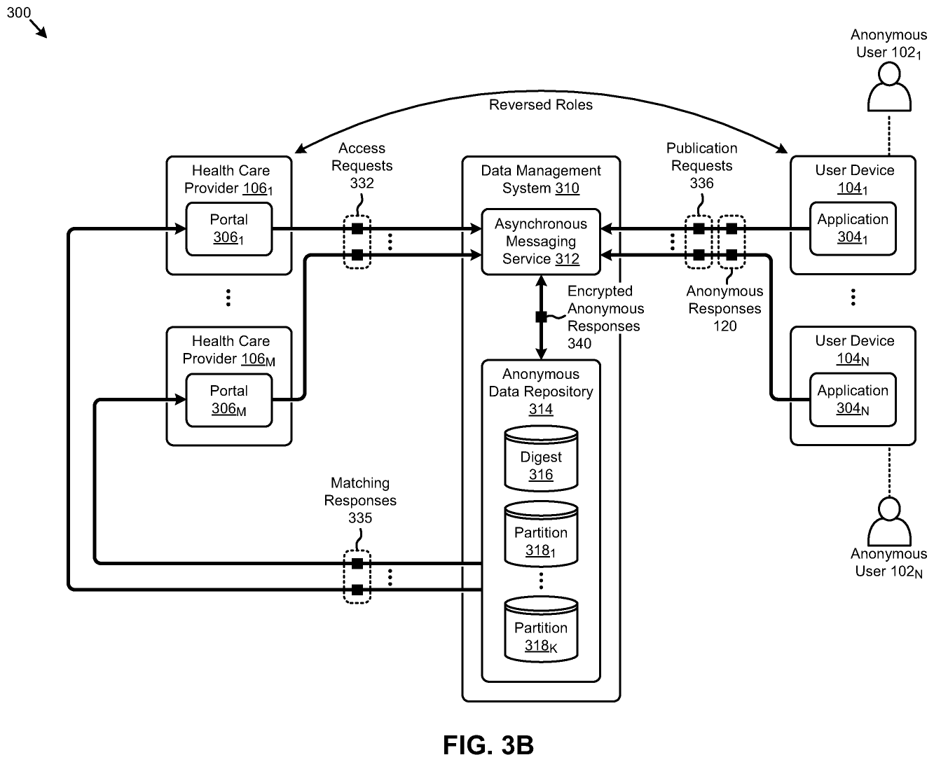

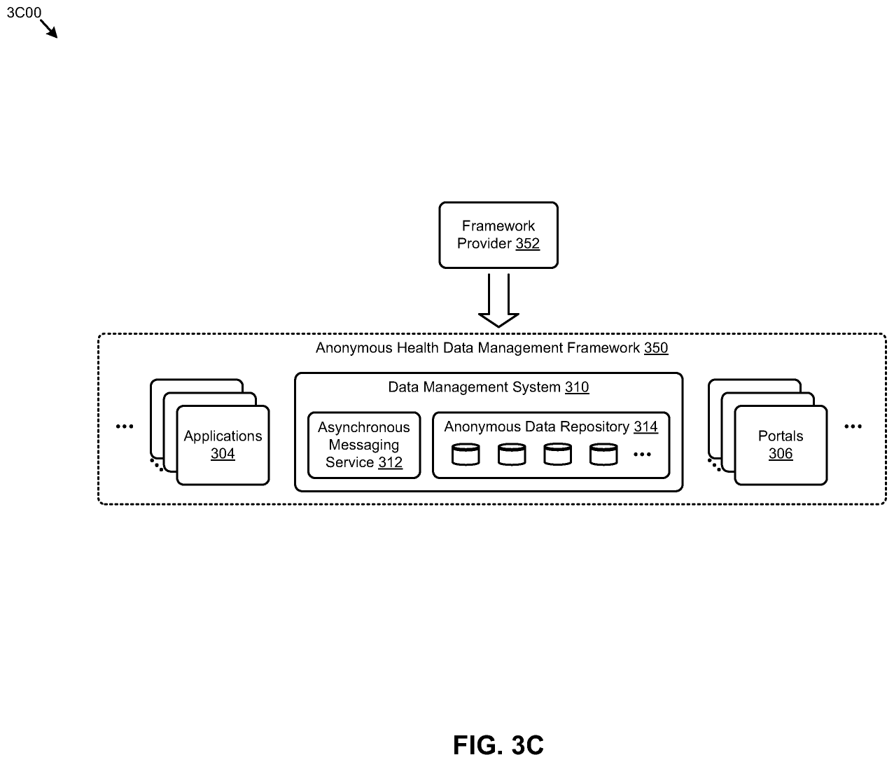

FIG. 3A and FIG. 3B depict a system 300 that implements deniable digital health diagnoses. As an option, one or more variations of system 300 or any aspect thereof may be implemented in the context of the architecture and functionality of the embodiments described herein. The system 300 or any aspect thereof may be implemented in any environment.

FIG. 3A and FIG. 3B illustrate aspects pertaining to implementing an anonymous health data management framework to secretly match a particular anonymous user to his or her anonymous health data that is published for access only by the particular anonymous user. Specifically, the figures are being presented to show one embodiment of certain representative computing system components, together with associated data structures and associated data flows, that are implemented to facilitate the herein disclosed techniques. The components, data flows, and data structures shown in FIG. 3A and FIG. 3B present merely one partitioning and merely one data manipulation approach. As such, the specific examples shown are purely illustrative, and other components, subsystems, data structures, data flows, and/or partitioning are reasonable.

As shown, system 300 comprises an instance of a data management system 310 to manage the exchange of anonymous health data between participants in a digital health ecosystem. An asynchronous messaging service 312 at data management system 310 exposes various endpoints to the participants that facilitate certain interactions with an anonymous data repository 314. For example, asynchronous messaging service 312 facilitates asynchronous instances of publication requests 336 and access requests 332 to publish instances of anonymous health data entries 118 to anonymous data repository 314 and retrieves instances of matching health data entries 334 from anonymous data repository 314, respectively.

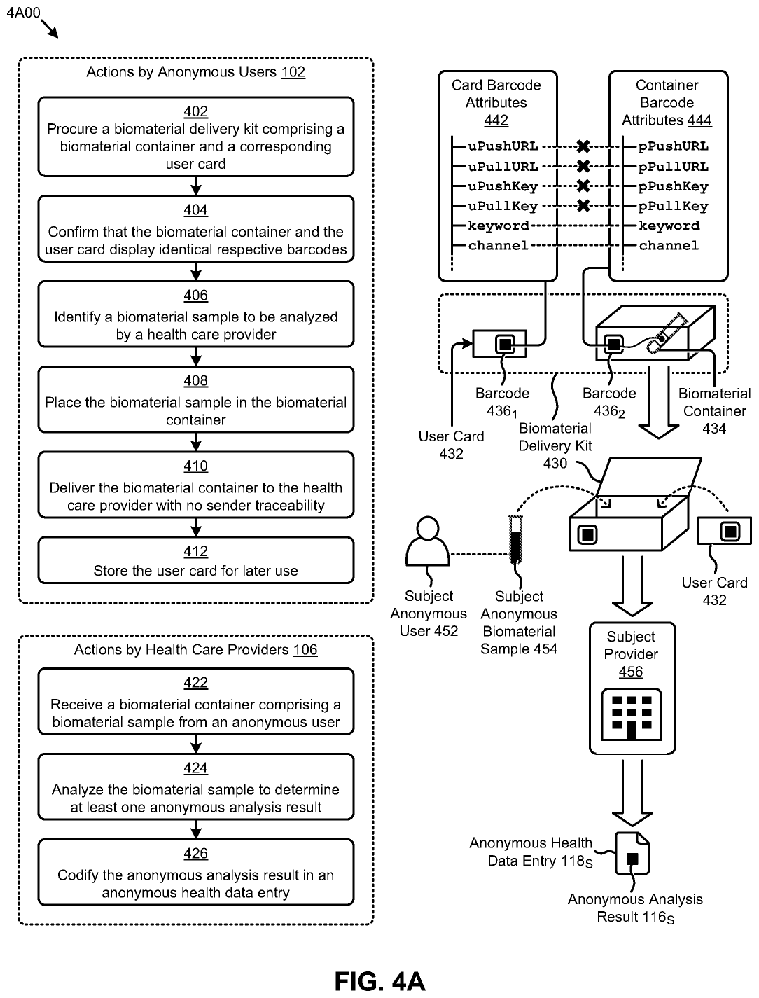

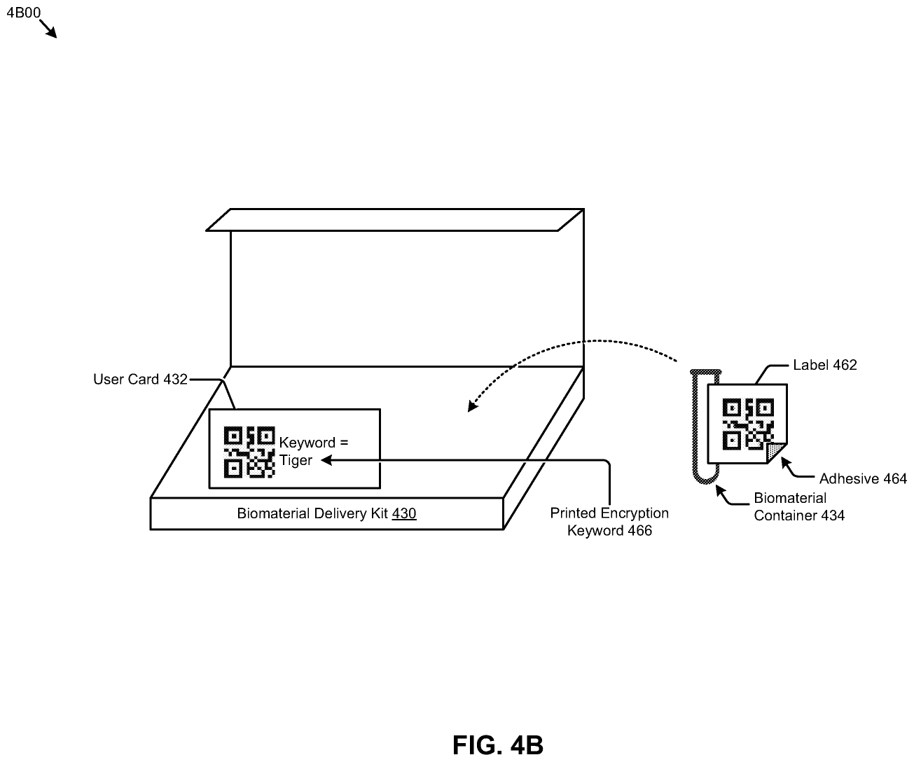

In certain embodiments, certain portions of data management system 310 are configured to operate according to a publish-subscribe model. In accordance with the publish-subscribe model, asynchronous messaging service 312 facilitates the publication of anonymous health data entries 118 to various partitions (e.g., partition 318₁, . . . , partition 318_{K}) of anonymous data repository 314. The partitions might correspond, for example, to a channel associated with a particular entry, which channels are described in more detail as pertains to FIG. 4A. The partitions are often configured to reduce the latency and/or computing resource consumption when accessing the anonymous health data entries 118 published to anonymous data repository 314.

Specifically, the partitions might expose respective endpoints (e.g., service URL) to the participants in the digital health ecosystem to direct each participant to scan a particular partition to “pull” matching instances of matching health data entries 334 rather than scan the entire anonymous data repository. In this case, the partitions are populated with a number of anonymous health data entries to avoid non-negligible digital traces being associated with the participants. For example, a participant that scans a partition is more likely associated with any one of the anonymous health data entries stored in a partition when there are merely a few (e.g., 10) entries in that partition, but less likely associated with any one of the entries in the partition when there are a large number (e.g., 10,000) of entries in that partition. In some cases, a particular partition might be initially intentionally populated with a large number of fake instances of anonymous health data entries to achieve the aforementioned negligible digital trace for participants who access that partition.

In some cases, all samples of all user participants undergo the same suite of genetic tests and/or other tests under a subscription plan (e.g., a monthly plan). As such, since all participant samples that are subject samples in the aforementioned subscription plan are subjected to the same suite of tests, the type of test and/or combination of tests cannot be used to discern information from a health data entry that correlates to a particular user. Moreover, a particular partition that corresponds to a subscription plan might be initially intentionally populated with a large number of fake instances of anonymous health data entries.

As shown, a digest 316 is also present in anonymous data repository 314. Digest 316 comprises digest entries that correspond to respective instances of anonymous health data entries stored in the partitions of the repository. Such digest entries are configured to be more efficiently scanned by a participant so as to reduce the latency and/or computing resource consumption when pulling the anonymous health data entries 118 published to anonymous data repository 314. For example, the digest entries might be small summary files or data objects that merely reference the location of a respective anonymous health data entry and/or the location of the partition that stores the anonymous health data entry. Neither any digest entry nor any entry of any sort in any variation of the anonymous data repositories comprise any raw (e.g., unencrypted) user data. In some cases, Beaver triples are used to enforce various publish-subscribe model tenets that allow only the particular subscriber that corresponds to a particular published entry to unencrypt a published entry.

In any case, in accordance with the publish-subscribe model, participants in the digital health ecosystem that publish certain data repository entries are unaware of the participants that access the entries, and the participants that access the entries are unaware of the participants that publish the entries.

Further details regarding general approaches to use of Beaver triples in multi-party computation situations are described in U.S. application Ser. No. 16/537,523 titled “VERIFYING DATA ACCURACY IN PRIVACY-PRESERVING COMPUTATIONS”, filed on Aug. 9, 2019, which is hereby incorporated by reference in its entirety.

As depicted in FIG. 3A, such participants in a digital health ecosystem can include a plurality of anonymous users (e.g., anonymous user 102₁, . . . , anonymous user 102_{N}) and a plurality of health care providers (e.g., health care provider 106₁, . . . , health care provider 106_{M}). As can be observed, the health care providers receive collections of anonymous biomaterials 112 from the anonymous users for analysis. The respective sets of anonymous analysis results derived from anonymous biomaterials 112 are codified into respective instances of anonymous health data entries 118. The anonymous health data entries (e.g., PDF files) might be generated by the health care providers or by one or more other participants in the digital health ecosystem.

The health care providers (or other participants that generated the anonymous health data entries) interact with respective instances of a portal (e.g., portal 306₁, . . . , portal 306_{M}) to issue the publication requests 336 and anonymous health data entries 118 to asynchronous messaging service 312. As an example, the portals might be provider-specific instances of a web application that are served by data management system 310 and presented in respective instances of a browser at the health care providers.

Moreover, the anonymous users interact with instances of an application (e.g., application 304₁, . . . , application 304_{N}) operating on respective user devices (e.g., user device 104₁, . . . , user device 104_{N}) to issue the access requests 332 to asynchronous messaging service 312 and pull the matching health data entries 334 from anonymous data repository 314. The computing entities (e.g., applications, portals, etc.) used by the participants (e.g., anonymous users, health care providers, third parties, etc.) can be any entity capable of issuing messages (e.g., publication requests, access requests, content uploads or downloads, HTTPS requests, etc.) to asynchronous messaging service 312 and/or any other service implemented to carry out the herein disclosed techniques.

In the embodiment depicted in FIG. 3A, the instances of anonymous health data entries 118 are encrypted to generate instances of encrypted anonymous health data entries 338 that are stored in anonymous data repository 314. As shown, the aforementioned digest entries are also encrypted and stored as encrypted digest entries 339 in digest 316. Such encryption not only serves to protect the underlying information contained in the entries, but also facilitates the matching of the anonymous users with respective instances of anonymous health data entries. Specifically, and as described in further detail herein, a subject anonymous user can discover a matching digest entry or anonymous health data entry encrypted with a first key from a pair of keys by successfully decrypting the entry with a second key from the pair of keys.

As earlier mentioned, the roles of certain participants in the digital health ecosystem may be reversed and/or otherwise adopted by the participants. In FIG. 3B, the anonymous users take on the role of anonymous data publisher and the health care providers take on the role of anonymous data requestor. In this case, the anonymous users (e.g., anonymous user 102₁, . . . , anonymous user 102_{N}) who discover matching health data entries interact with applications (e.g., application 304₁, . . . , application 304_{N}) at their respective user devices (e.g., user device 104₁, . . . , user device 104_{N}) to issue the publication requests 336 and anonymous responses 120 to asynchronous messaging service 312. In response to the publication requests, asynchronous messaging service 312 publishes instances of encrypted anonymous responses 340 derived from respective instances of anonymous responses 120.

As with the aforementioned instances of encrypted anonymous health data entries 338 and encrypted digest entries 339 (as shown in FIG. 3A), encrypted anonymous responses 340 facilitate the matching of the published responses to their target recipients.

In the scenario of FIG. 3B, the target recipients for anonymous responses 120 and/or encrypted anonymous responses 340 published to anonymous data repository 314 are the health care providers (e.g., health care provider 106₁, . . . , health care providers 106_{M}). As such, the health care providers interact with respective portals (e.g., portal 306₁, . . . , portal 306_{M}) to issue the access requests 332 to asynchronous messaging service 312 at data management system 310 in order to pull matching responses 335 from anonymous data repository 314. As an example, a subject provider that published subject anonymous analysis results derived from anonymous biomaterials of a subject anonymous user will be matched with a subject anonymous response from the subject anonymous user.

The foregoing discussions include computing entities that may constitute an anonymous health data management framework that is implemented to facilitate the herein disclosed techniques, which framework is disclosed in further detail as follows.

FIG. 3C illustrates a health data management framework 3C00 that facilitates deniable digital health diagnoses. As an option, one or more variations of health data management framework 3C00 or any aspect thereof may be implemented in the context of the architecture and functionality of the embodiments described herein. The health data management framework 3C00 or any aspect thereof may be implemented in any environment.

FIG. 3C illustrates aspects pertaining to implementing an anonymous health data management framework to secretly match a particular anonymous user to his or her anonymous health data that is published for access only by the particular anonymous user. Specifically, the figure presents a logical depiction of representative components of an anonymous health data management framework that may be used to facilitate the herein disclosed techniques.

As shown, an anonymous health data management framework 350 comprises several of the earlier mentioned computing entities associated with a digital health ecosystem. Specifically, the framework comprises an instance of data management system 310 having an asynchronous messaging service 312 that, at least in part, manages the data (e.g., anonymous health data entries, digest entries, anonymous responses, etc.) stored in anonymous data repository 314. In certain embodiments, data management system 310 may operate over one or more participants in the digital health ecosystem or operate over one or more entities external to the digital health ecosystem.

The anonymous health data management framework 350 further comprises instances of applications 304, portals 306, and/or other computing entities that operate, for example, at the user devices of various participants in the digital health data ecosystem. As can be observed, anonymous health data management framework 350 may be implemented by framework provider 352. For example, framework provider 352 might be the owner and operator of data management system 310 that also develops and delivers (e.g., for local installation, local browser access, etc.) the instances of applications 304 and portals 306 to interact with data management system 310.

The foregoing discussions include techniques for delivering anonymous biomaterials from anonymous users to be processed (e.g., analyzed) by health care providers in a digital health ecosystem, which techniques are disclosed in further detail as follows.

FIG. 4A presents an anonymous biomaterials processing technique 4A00 as implemented in systems that facilitate deniable digital health diagnoses. As an option, one or more variations of anonymous biomaterials processing technique 4A00 or any aspect thereof may be implemented in the context of the architecture and functionality of the embodiments described herein. The anonymous biomaterials processing technique 4A00 or any aspect thereof may be implemented in any environment.

FIG. 4A illustrates aspects pertaining to implementing an anonymous health data management framework to secretly match a particular anonymous user to his or her anonymous health data that is published for access only by the particular anonymous user. Specifically, the figure is presented to illustrate one embodiment of certain steps and/or operations that facilitate the delivery of anonymous biomaterials from anonymous users for processing (e.g., analyzing) by health care providers in a digital health ecosystem. As depicted in the figure, respective portions of the steps and/or operations are performed by the anonymous users (e.g., anonymous user 102) and the health care providers (e.g., health care providers 106). A representative scenario is also shown in the figure to illustrate an example application of anonymous biomaterials processing technique 4A00.

Anonymous biomaterials processing technique 4A00 commences by one or more of the anonymous users 102 procuring a biomaterial collection and delivery kit that comprises a biomaterial container and a corresponding user card (step 402). As illustrated, a biomaterial delivery kit 430 comprises a biomaterial container 434 and a user card 432. The user can collect biomaterials (e.g., hair, epithelial cells, urine, etc.) and deposit the biomaterials into the biomaterial container, which can then be mailed anonymously either using the delivery kit container itself or a separate mailer (not shown).

To verify the correspondence between the printing on the user card and the identification of the biomaterial container or separate mailer, the display of an identical set of identification codes (e.g., barcodes, two-dimensional QR codes) on the biomaterial container and the user card are confirmed (step 404). A barcode or QR code scanning function of an application (e.g., an app on a user device having an image sensor) can be used to confirm that the code of the biomaterial container and the code of the user card are identical.

As shown, a barcode 436₁ is displayed on user card 432 and a barcode 436₂ is displayed on biomaterial container 434. In some cases, barcode 436₁ may be displayed inside the biomaterial container 434 and/or on another container (e.g., vial, test tube, etc.) that is stored in biomaterial container 434. As used herein, a barcode is set of visual symbols, which symbols present a machine-readable representation of data. One variant of a barcode is a quick response code or QR code. The QR code is a two-dimensional barcode that can represent more data in a particular space than a standard linear barcode. These two-dimensional QR codes can carry sufficient extra bits of information that can be used for error detection as well as error correction. As such, a QR code can be scanned by an app on a user device having an image sensor (e.g., camera), and the app can verify that all of the bits of the code were scanned and decoded error-free.

As indicated by a set of card barcode attributes 442 and a set of container barcode attributes 444, certain respective attributes are described by the data of barcode 436₁ and the data of barcode 436₂ to facilitate at least some embodiments of the herein disclosed techniques. Specifically, card barcode attributes 442 indicate that the data of barcode 436₁ might describe a user publication service endpoint (e.g., associated with a “uPushURL” field), a user request service endpoint (e.g., associated with a “uPullURL” field), a user data publication key (e.g., associated with a “uPushKey” field), a user data request key (e.g., associated with a “uPullKey”), an encryption keyword (e.g., associated with a “keyword” field), a publication channel (e.g., associated with a “channel” field), and/or other attributes.

Moreover, container barcode attributes 444 indicate that the data of barcode 436₂ might describe a provider publication service endpoint (e.g., associated with a “pPushURL” field), a provider request service endpoint (e.g., associated with a “pPullURL” field), a provider data publication key (e.g., associated with a “pPushKey” field), a provider data request key (e.g., associated with a “pPullKey”), an encryption keyword (e.g., associated with a “keyword” field), a publication channel (e.g., associated with a “channel” field), and/or other attributes. As can be observed, while the service endpoints and keys of barcode 436₁ and barcode 436₂ are different, the “keyword” and “channel” attributes of the barcodes may be the same. As an example, the “keyword” may be included in encrypted entries to facilitate quickly determining if an entry decryption attempt is successful. Furthermore, the “channel” attribute may be used to determine a target partition of a data repository and facilitate efficient discovery of the target partition.

When the biomaterial delivery kit has been procured and the barcodes confirmed, a biomaterial sample to be analyzed by a health care provider is identified (step 406) and placed into the biomaterial container (step 408). In the illustrated scenario, a collection of subject anonymous biomaterial sample 454 from a subject anonymous user 452 is placed into biomaterial container 434. The biomaterial container is then delivered to the health care provider with no sender traceability (step 410). The biomaterial container is delivered with no sender traceability to protect the anonymity of subject anonymous user 452 and subject anonymous biomaterial sample 454, at least from the perspective of the health care provider receiving the subject anonymous biomaterial sample 454. The user card corresponding to the delivered biomaterial container is stored for later use (e.g., by subject anonymous user 452) (step 412).

A biomaterial container comprising a biomaterial sample delivered from an anonymous user is received at one or more of the health care providers 106 (step 422). For example, biomaterial container 434 comprising the subject anonymous biomaterial sample 454 might be received by a subject provider 456 from health care providers 106. The biomaterial sample is analyzed to determine at least one anonymous analysis result (step 424) which is codified into an anonymous health data entry (step 426). As can be observed, an anonymous analysis result 116_{S} derived from the subject anonymous biomaterial sample 454 is codified into an anonymous health data entry 118_{S}. For example, anonymous health data entry 118_{S} might be a document that contains a description of the anonymous analysis result 116_{S}.

The foregoing discussions include techniques for using a biomaterials delivery kit. One embodiment of such a biomaterials delivery kit and preparation thereof is disclosed in further detail in FIG. 4B.

FIG. 4B presents a biomaterials delivery kit preparation technique 4B00 as implemented in systems that facilitate deniable digital health diagnoses.

As shown, biomaterial delivery kit 430 is a collection of several components. In this particular embodiment, a label 462 having printed thereon a unique code is affixed (e.g., using an adhesive 464) to an empty biomaterial container 434. The same unique code is also printed onto a user card 432, which may also include a printed encryption keyword 466. In the shown embodiment, the labeled empty biomaterial container and the user card are disposed into a box or other packaging. The box or other packaging is constructed and sealed such that the contents cannot be viewed by anyone other than an anonymous purchaser. More specifically, due to the materials, construction, and sealing techniques used in formation of the biomaterial delivery kit, evidence of tampering would be apparent to a would-be purchaser such that the would-be purchaser can choose to purchase a different unit of the biomaterial delivery kit—one that does not exhibit evidence of tampering.

There are many variations of a biomaterial delivery kits. Exemplary embodiments can comprise merely a biomaterial container having an association to an identification code and some form of tangible media (e.g., a user card) that has printed thereon the identification code. Even though the biomaterial container is configured with an association to the identification code, neither the code nor the container has any discernible association with a particular user. More specifically, the code does not include any personally identifiable information, and the code does not include any patient health information. The anonymous user can send the biomaterial container with its contents to a provider after which the contents of the biomaterial container is used to generate anonymous analysis results. The anonymous analysis results are published to a data repository that comprises anonymous health data entries. By operation of the data repository itself, or by operation of an access technique (e.g., a software routine), an access request comprising the identification code is matched to at least one of the anonymous health data entries. As such, anonymity of the user of the biomaterial delivery kit is protected. In some cases, a biomaterial delivery kit includes a mailer (e.g., envelope) that has a preprinted destination address as well as a preprinted return address. In some cases, the preprinted return address indicates a dead letter address. In some cases, a biomaterial delivery kit itself serves as a mailer that has a preprinted destination address as well as a preprinted return address. In some cases, the mailer has a double wrapping such that after depositing the sample into the mailer, the outer wrapper can be removed—thus eliminating the possibility that the user can leave traces of his or her identity (e.g., fingerprints) on the mailer itself. In these and other cases, the anonymity of the user of the biomaterial delivery kit is preserved.

The foregoing discussions include techniques for delivery of anonymous biomaterials to one or more health care providers, which health care providers publish anonymous analysis results to a data repository (e.g., step 214 of FIG. 2). Several variations of such publication techniques are disclosed in further detail as follows.

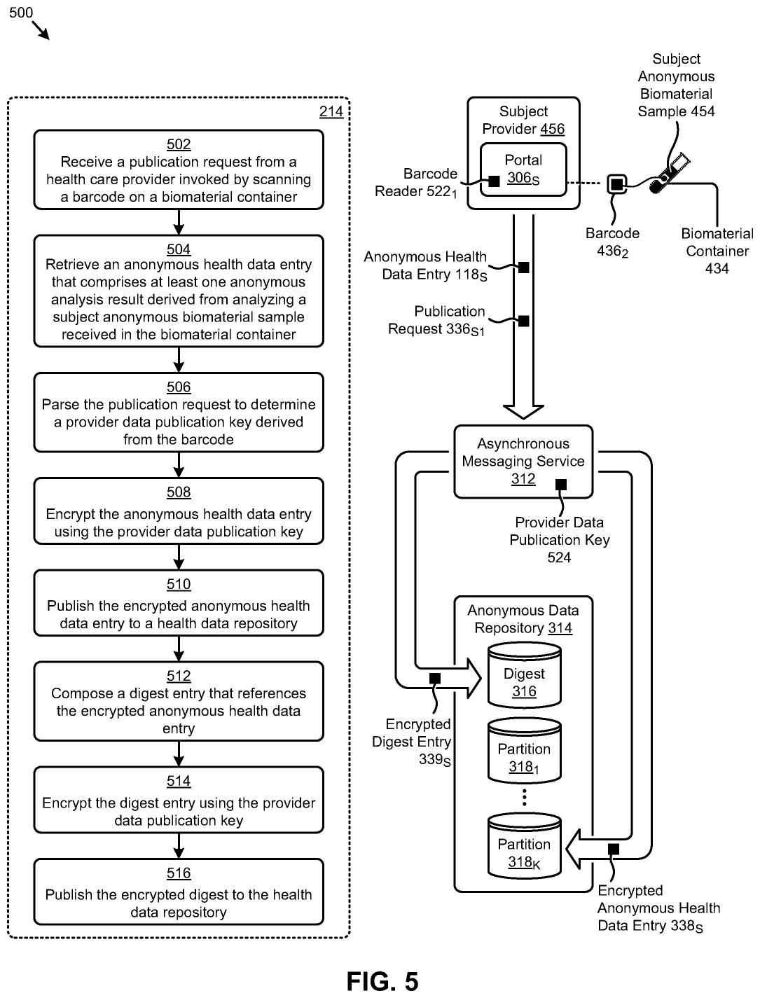

FIG. 5 depicts an anonymous health data publication technique 500 as implemented in systems that facilitate deniable digital health diagnoses. As an option, one or more variations of anonymous health data publication technique 500 or any aspect thereof may be implemented in the context of the architecture and functionality of the embodiments described herein. The anonymous health data publication technique 500 or any aspect thereof may be implemented in any environment.

FIG. 5 illustrates aspects pertaining to implementing an anonymous health data management framework to secretly match a particular anonymous user to his or her anonymous health data that is published for access only by the particular anonymous user. Specifically, the figure is presented to illustrate one embodiment of certain steps and/or operations that facilitate publishing anonymous analysis results and/or anonymous health data entries comprising anonymous analysis results to an anonymous data repository. As depicted in the figure, the steps and/or operations are associated with step 214 of FIG. 2. A representative scenario is also shown in the figure to illustrate an example application of anonymous health data publication technique 500.

Anonymous health data publication technique 500 commences by receiving a publication request from a health care provider invoked by scanning a barcode on a biomaterial container (step 502). As illustrated, subject provider 456 might invoke a publication request 336_{S1} to asynchronous messaging service 312 by scanning the barcode 436₂ on biomaterial container 434 using a barcode reader 522₁ associated with portal 306_{S}. For example, barcode reader 522₁ might use a camera on a smart phone, a web cam on a laptop or desktop computer, a barcode scanning device, or another mechanism that communicates with portal 306_{S} to scan the barcode 436₂. An anonymous health data entry that comprises at least one anonymous analysis result derived from analyzing the subject anonymous biomaterial sample 454 is retrieved (step 504). As an example, in response to receiving the publication request 336_{S1}, asynchronous messaging service 312 issues a response to portal 306_{S} associated with subject provider 456 requesting upload of anonymous health data entry 118_{S} to the service.

The publication request is parsed to determine a provider data publication key derived from the barcode (step 506). For example, asynchronous messaging service 312 parses the payload of publication request 336_{S1} to determine a provider data publication key 524 derived from barcode 436₂. The anonymous health data entry is encrypted using the provider data publication key (step 508) and the encrypted anonymous health data entry is published to a health data repository (step 510). As illustrated, an encrypted anonymous health data entry 338_{S} generated by encrypting the anonymous health data entry 118_{S} with provider data publication key 524 is published to anonymous data repository 314. The particular partition (e.g., partition 318_{K}) of anonymous data repository 314 that is identified for storing the encrypted anonymous health data entry 338_{S} may be identified based at least in part on a publication channel derived from barcode 436₂. In some cases, an encryption keyword derived from barcode 436₂ is included in encrypted anonymous health data entry 338_{S} to facilitate quickly determining if an attempt to decrypt the entry is successful.

Furthermore, in some cases, a digest entry that references the encrypted anonymous health data entry is composed (step 512). The digest entry is encrypted with the provider data publication key (step 514) and published to the health data repository (step 516). As shown, an encrypted digest entry 339_{S} that references the encrypted anonymous health data entry 338_{S} is published to digest 316 of anonymous data repository 314. The digest entry underlying the encrypted digest entry 339_{S} might be a small summary file or data object that references the location (e.g., in partition 318_{K} of anonymous data repository 314) of encrypted anonymous health data entry 338_{S}. In some cases, an encryption keyword derived from barcode 436₂ is included in encrypted digest entry 339_{S} to facilitate quickly determining if an attempt to decrypt the entry is successful.

The foregoing discussions include techniques for processing access requests to match anonymous users with respective anonymous health data (e.g., analysis results) and delivering the matching health data to the users (e.g., step 222 and step 224 of FIG. 2), which techniques are disclosed in further detail as follows.

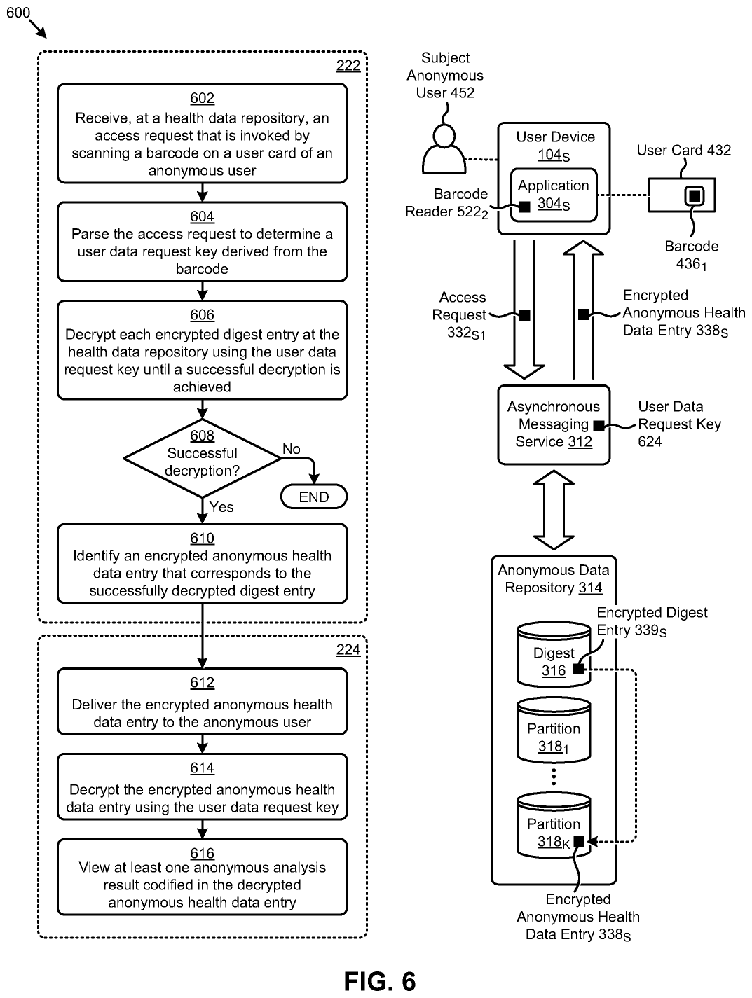

FIG. 6 presents an anonymous health data access technique 600 as implemented in systems that facilitate deniable digital health diagnoses. As an option, one or more variations of anonymous health data access technique 600 or any aspect thereof may be implemented in the context of the architecture and functionality of the embodiments described herein. The anonymous health data access technique 600 or any aspect thereof may be implemented in any environment.

FIG. 6 illustrates aspects pertaining to implementing an anonymous health data management framework to secretly match a particular anonymous user to his or her anonymous health data that is published for access only by the particular anonymous user. Specifically, the figure is presented to illustrate one embodiment of certain steps and/or operations that facilitate processing access requests to match anonymous users with respective anonymous health data (e.g., analysis results) and delivering the matching health data to the users. As depicted in the figure, the steps and/or operations are associated with step 222 and step 224 of FIG. 2. A representative scenario is also shown in the figure to illustrate an example application of anonymous health data access technique 600.

Anonymous health data access technique 600 commences by receiving at a health data repository an access request from an anonymous user that is invoked by scanning a barcode on a user card (step 602). As illustrated, subject anonymous user 452 might invoke an access request 332_{S1} to the asynchronous messaging service 312 managing the anonymous data repository 314 by scanning the barcode 436₁ on user card 432 using a barcode reader 522₂ associated with application 304_{S} operating on user device 104_{S}. For example, barcode reader 522₂ might use a camera on user device 104_{S} (e.g., a smart phone, a laptop computer, a desktop computer, etc.), a barcode scanning device, or another mechanism that communicates with application 304_{S} to scan the barcode 436₁. The access request is parsed to determine a user data request key derived from the barcode (step 604). For example, asynchronous messaging service 312 parses the payload of access request 332_{S1} to determine a user data request key 624 derived from barcode 436₁.

Each encrypted digest entry at the health data repository is decrypted until a successful decryption is achieved (step 606). For example, asynchronous messaging service 312 traverses the encrypted digest entries stored in digest 316 of anonymous data repository 314 and decrypts each in turn until a successful decryption is achieved. In some cases, a successful decryption is indicated by the discovery of a readable encryption keyword embedded in the digest entry at the time of encryption. If a successful encryption is not achieved (“No” path of decision 608), no further actions are performed in accordance with anonymous health data access technique 600. In some cases, when a successful decryption is not achieved, a message is submitted to the issuer (e.g., subject anonymous user 452) of the access request indicating that a matching entry is not found in the repository.

If a successful decryption is achieved (“Yes” path of decision 608), an encrypted anonymous health data entry 338_{S} that corresponds to the successfully decrypted digest entry is identified (step 610). As an example, asynchronous messaging service 312 might successfully decrypt the encrypted digest entry 339_{S} in digest 316 of anonymous data repository 314 using the user data request key 624 associated with access request 332_{S1} from subject anonymous user 452. As illustrated, the decrypted instance of encrypted digest entry 339_{S} can then be analyzed to discover a reference included in the digest entry that identifies the encrypted anonymous health data entry 338_{S}.

The encrypted anonymous health data entry referenced by the decrypted digest entry is delivered to the anonymous user (step 612). As can be observed, encrypted anonymous health data entry 338_{S} is delivered by asynchronous messaging service 312 to application 304_{S} at user device 104_{S}. The encrypted anonymous health data entry is decrypted using the user data request key (step 614) to facilitate viewing of the at least one anonymous analysis result codified in the decrypted anonymous health data entry (step 616). According to the scenario illustrated in the figure, encrypted anonymous health data entry 338_{S} is decrypted at application 304_{S} using the user data request key 624 derived from barcode 436₁ to view at least one anonymous analysis result recorded in the anonymous health data entry.

The foregoing discussions include techniques for publishing anonymous responses to a data repository (e.g., step 218 of FIG. 2), which techniques are disclosed in further detail as follows.

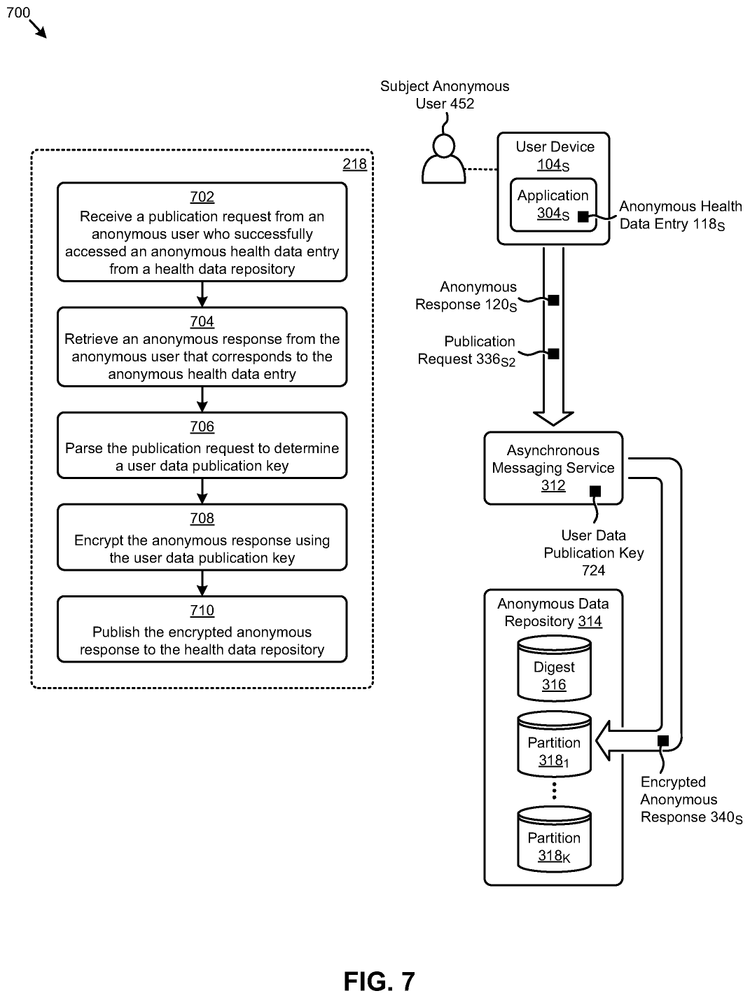

FIG. 7 depicts an anonymous response publication technique 700 as implemented in systems that facilitate deniable digital health diagnoses. As an option, one or more variations of anonymous response publication technique 700 or any aspect thereof may be implemented in the context of the architecture and functionality of the embodiments described herein. The anonymous response publication technique 700 or any aspect thereof may be implemented in any environment.

FIG. 7 illustrates aspects pertaining to implementing an anonymous health data management framework to secretly match a particular anonymous user to his or her anonymous health data that is published for access only by the particular anonymous user. Specifically, the figure is presented to illustrate one embodiment of certain steps and/or operations that facilitate publishing anonymous responses to an anonymous data repository accessible by certain participants in a digital health ecosystem. As depicted in the figure, the steps and/or operations are associated with step 218 of FIG. 2. A representative scenario is also shown in the figure to illustrate an example application of anonymous response publication technique 700.

Anonymous response publication technique 700 commences by receiving a publication request from an anonymous user who successfully accessed an anonymous health data entry at a health data repository (step 702). As illustrated, asynchronous messaging service 312 might receive a publication request 336_{S2} invoked by subject anonymous user 452 from application 304_{S} at user device 104_{S} in response to accessing the anonymous health data entry 118_{S}. An anonymous response that corresponds to the anonymous health data entry is retrieved (step 704). As an example, an anonymous response 120_{S} that comprises a question about the results in anonymous health data entry 118_{S} might be submitted by subject anonymous user 452 and retrieved by asynchronous messaging service 312 in response to receiving the publication request 336_{S2}.

The publication request is parsed to determine a user data publication key (step 706). For example, asynchronous messaging service 312 parses the payload of publication request 336_{S2} to determine a user data publication key 724. In some cases, user data publication key 724 might be derived from a barcode displayed on a user card held by subject anonymous user 452. The anonymous response is encrypted using the user data publication key (step 708) and the encrypted anonymous response is published to a health data repository (step 710). As illustrated, an encrypted anonymous response 340_{S} generated by encrypting the anonymous response 120_{S} with user data publication key 724 is published to anonymous data repository 314. The particular partition (e.g., partition 318₁) of anonymous data repository 314 that is identified for storing the encrypted anonymous response 340_{S} may be identified based at least in part on a publication channel (e.g., derived from a barcode), a data content type (e.g., response, analysis results, digest, etc.), and/or other characteristics. In some cases, an encryption keyword (e.g., derived from a barcode) is included in encrypted anonymous response 340_{S} to facilitate quickly determining if an attempt to decrypt the response is successful.

The foregoing discussions include techniques for processing access requests to match health care providers with respective anonymous responses from anonymous users and then delivering the matching responses to the providers (e.g., step 226 and step 228 of FIG. 2), which techniques are disclosed in further detail as follows.

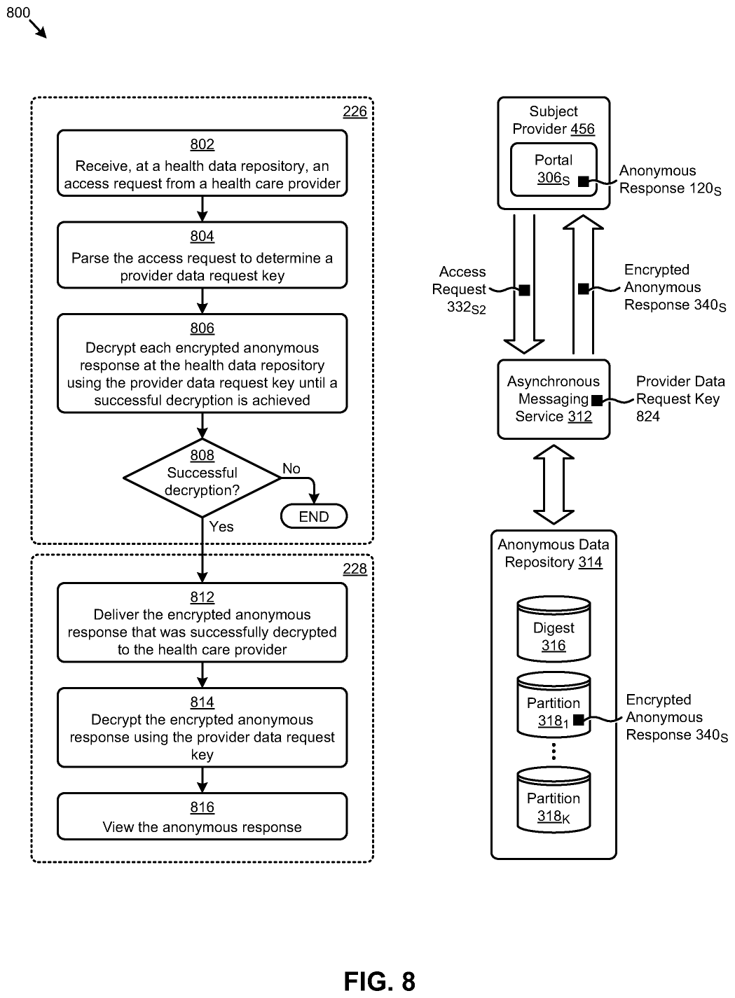

FIG. 8 presents an anonymous response access technique 800 as implemented in systems that facilitate deniable digital health diagnoses. As an option, one or more variations of anonymous response access technique 800 or any aspect thereof may be implemented in the context of the architecture and functionality of the embodiments described herein. The anonymous response access technique 800 or any aspect thereof may be implemented in any environment.

FIG. 8 illustrates aspects pertaining to implementing an anonymous health data management framework to secretly match a particular anonymous user to his or her anonymous health data that is published for access only by the particular anonymous user. SPECIFICALLY, the figure is presented to illustrate one embodiment of certain steps and/or operations that facilitate processing access requests to match one or more health care providers with respective anonymous responses from anonymous users and then delivering the matching responses to the providers. As depicted in the figure, the steps and/or operations are associated with step 226 and step 228 of FIG. 2. A representative scenario is also shown in the figure to illustrate an example application of anonymous response access technique 800.

Anonymous response access technique 800 commences by receiving at a health data repository an access request from a health care provider (step 802). As illustrated, subject provider 456 might invoke an access request 332_{S2} from portal 306_{S} to the asynchronous messaging service 312 managing the anonymous data repository 314. Subject provider 456 might be one of many health care providers and/or other participants in a digital health ecosystem that are issuing access requests to anonymous data repository 314 to discover respective anonymous responses published for access by the providers and/or participants. The access request is parsed to determine a provider data request key (step 804). For example, asynchronous messaging service 312 parses the payload of access request 332_{S2} to determine a provider data request key 824. In some cases, provider data request key 824 might be derived from a barcode displayed on a biomaterial container in the possession of subject provider 456.

Each encrypted anonymous response at the health data repository is decrypted until a successful decryption is achieved (step 806). For example, asynchronous messaging service 312 traverses the encrypted anonymous responses stored in anonymous data repository 314 and decrypts each one until a successful decryption is achieved. In some cases, a successful decryption is indicated by the discovery of a readable encryption keyword embedded in the anonymous response at the time of encryption. If a successful encryption is not achieved (“No” path of decision 808), no further actions are performed in accordance with anonymous response access technique 800. In some cases, when a successful decryption is not achieved, a message is submitted to the issuer (e.g., health care provider) of the access request indicating that a matching response is not found in the repository.

If a successful decryption is achieved (“Yes” path of decision 808), the encrypted anonymous response is delivered to the health care provider (step 812). As can be observed, encrypted anonymous response 340_{S} from partition 318₁ of anonymous data repository 314 is delivered by asynchronous messaging service 312 to portal 306_{S} at subject provider 456. The encrypted anonymous response is decrypted using the provider data request key (step 814) to facilitate viewing of the anonymous response (step 816). According to the scenario illustrated in the figure, encrypted anonymous response 340_{S} is decrypted using the provider data request key 824 to view the anonymous response 120_{S} at portal 306_{S}.

### Additional Embodiments of the Disclosure

### Additional Practical Application Examples

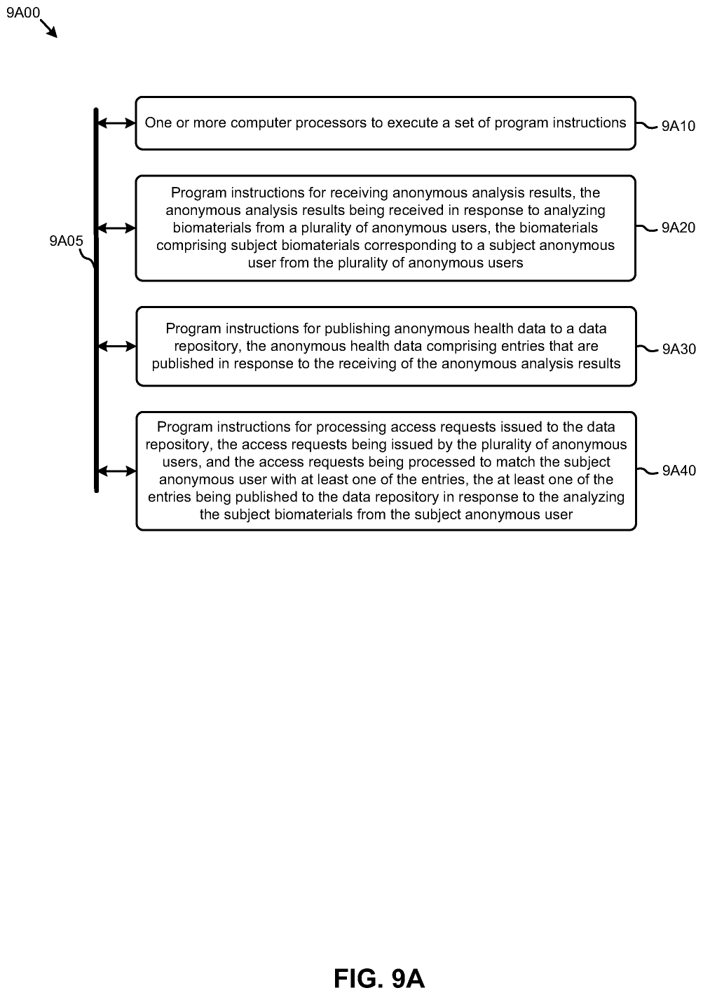

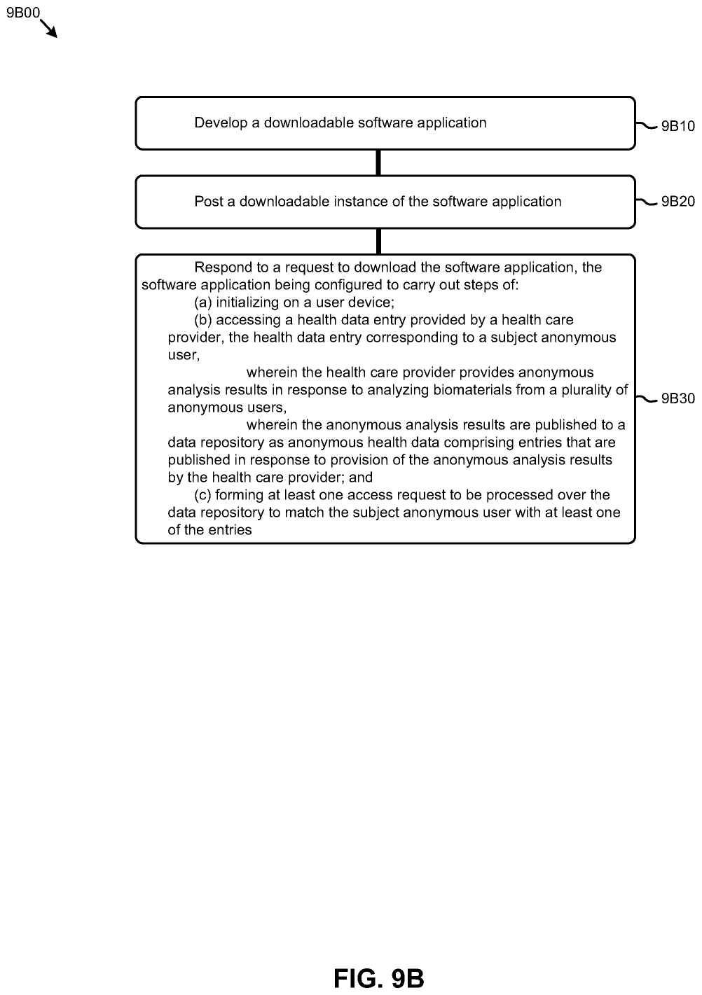

FIG. 9A depicts a system 9A00 as an arrangement of computing modules that are interconnected so as to operate cooperatively to implement certain of the herein-disclosed embodiments. This and other embodiments present particular arrangements of elements that, individually or as combined, serve to form improved technological processes that address delivering diagnoses and/or other health information to anonymous patients in a digital health ecosystem. The partitioning of system 9A00 is merely illustrative and other partitions are possible. As an option, the system 9A00 may be implemented in the context of the architecture and functionality of the embodiments described herein. Of course, however, the system 9A00 or any operation therein may be carried out in any desired environment.

The system 9A00 comprises at least one processor and at least one memory, the memory serving to store program instructions corresponding to the operations of the system. As shown, an operation can be implemented in whole or in part using program instructions accessible by a module. The modules are connected to a communication path 9A05, and any operation can communicate with any other operations over communication path 9A05. The modules of the system can, individually or in combination, perform method operations within system 9A00. Any operations performed within system 9A00 may be performed in any order unless as may be specified in the claims.

The shown embodiment implements a portion of a computer system, presented as system 9A00, comprising one or more computer processors to execute a set of program code instructions (module 9A10) and modules for accessing memory to hold program code instructions to perform: receiving anonymous analysis results, the anonymous analysis results being received in response to analyzing biomaterials from a plurality of anonymous users, the biomaterials comprising subject biomaterials corresponding to a subject anonymous user from the plurality of anonymous users (module 9A20); publishing anonymous health data to a data repository, the anonymous health data comprising entries that are published in response to the receiving of the anonymous analysis results (module 9A30); processing access requests issued to the data repository, the access requests being issued by the plurality of anonymous users, and the access requests being processed to match the subject anonymous user with at least one of the entries, the at least one of the entries being published to the data repository in response to the analyzing the subject biomaterials from the subject anonymous user (module 9A40); and accessing the at least one of the entries corresponding to the subject anonymous user (module 9A50).

Variations of the foregoing may include more or fewer of the shown modules. Certain variations may perform more or fewer (or different) steps and/or certain variations may use data elements in more, or in fewer, or in different operations.

Still further, some embodiments include variations in the operations performed, and some embodiments include variations of aspects of the data elements used in the operations.

The system of FIG. 9B implements a method for delivering deniable digital health diagnoses. The shown steps include developing a downloadable software application (box 9B10); posting a downloadable instance of the software application (box 9B20); and responding to a request to download the software application, the software application being configured to carry out steps of: (a) initializing on a user device; (b) accessing a health data entry provided by a health care provider, the health data entry corresponding to a subject anonymous user, wherein the health care provider provides anonymous analysis results in response to analyzing biomaterials from a plurality of anonymous users, wherein the anonymous analysis results are published to a data repository as anonymous health data comprising entries that are published in response to provision of the anonymous analysis results by the health care provider; and (c) forming at least one access request to be processed over the data repository to match the subject anonymous user with at least one of the entries (box 9B30).

### System Architecture Overview

### Additional System Architecture Examples

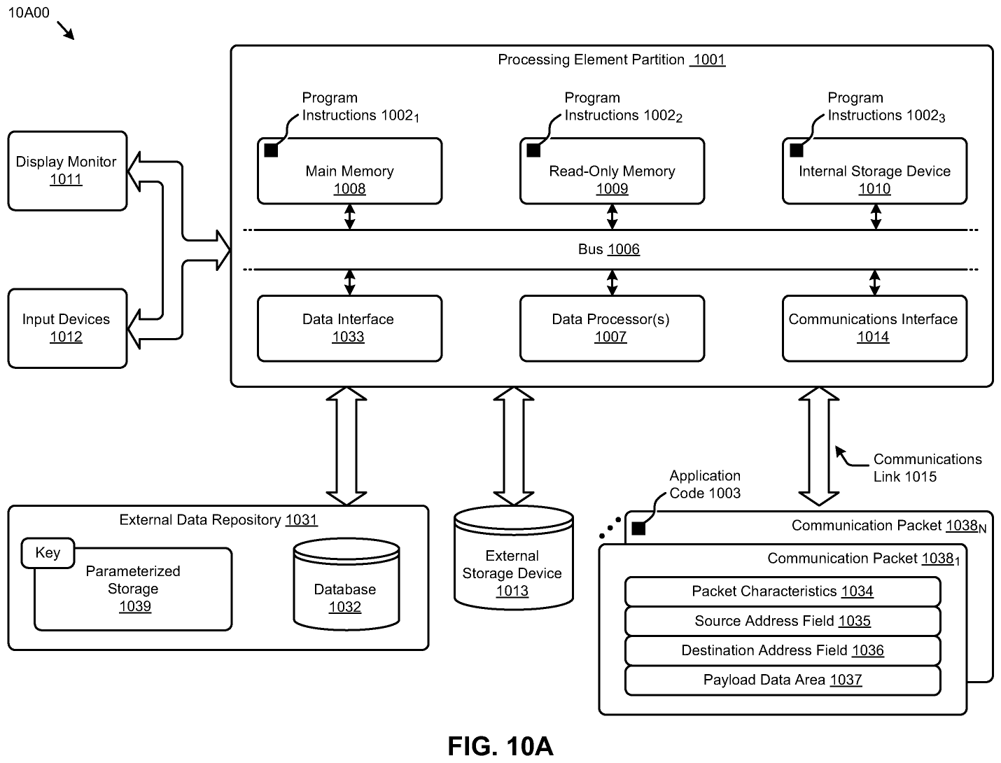

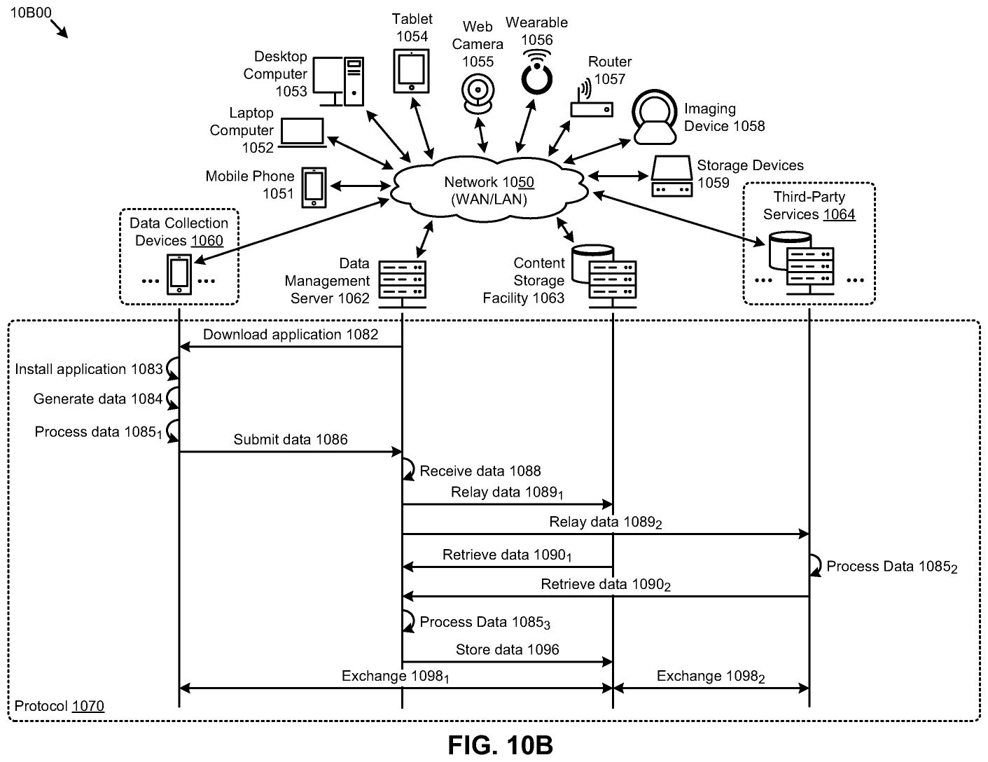

FIG. 10A depicts a block diagram of an instance of a computer system 10A00 suitable for implementing embodiments of the present disclosure. Computer system 10A00 includes a bus 1006 or other communication mechanism for communicating information. The bus interconnects subsystems and devices such as a central processing unit (CPU), or a multi-core CPU (e.g., data processor 1007), a system memory (e.g., main memory 1008, or an area of random access memory (RAM)), a non-volatile storage device or non-volatile storage area (e.g., read-only memory 1009), an internal storage device 1010 or external storage device 1013 (e.g., magnetic or optical), a data interface 1033, a communications interface 1014 (e.g., PHY, MAC, Ethernet interface, modem, etc.). The aforementioned components are shown within processing element partition 1001, however other partitions are possible. Computer system 10A00 further comprises a display 1011 (e.g., CRT or LCD), various input devices 1012 (e.g., keyboard, cursor control), and an external data repository 1031.

According to an embodiment of the disclosure, computer system 10A00 performs specific operations by data processor 1007 executing one or more sequences of one or more program instructions contained in a memory. Such instructions (e.g., program instructions 1002₁, program instructions 1002₂, program instructions 1002₃, etc.) can be contained in or can be read into a storage location or memory from any computer readable/usable storage medium such as a static storage device or a disk drive. The sequences can be organized to be accessed by one or more processing entities configured to execute a single process or configured to execute multiple concurrent processes to perform work. A processing entity can be hardware-based (e.g., involving one or more cores) or software-based, and/or can be formed using a combination of hardware and software that implements logic, and/or can carry out computations and/or processing steps using one or more processes and/or one or more tasks and/or one or more threads or any combination thereof.

According to an embodiment of the disclosure, computer system 10A00 performs specific networking operations using one or more instances of communications interface 1014. Instances of communications interface 1014 may comprise one or more networking ports that are configurable (e.g., pertaining to speed, protocol, physical layer characteristics, media access characteristics, etc.) and any particular instance of communications interface 1014 or port thereto can be configured differently from any other particular instance. Portions of a communication protocol can be carried out in whole or in part by any instance of communications interface 1014, and data (e.g., packets, data structures, bit fields, etc.) can be positioned in storage locations within communications interface 1014, or within system memory, and such data can be accessed (e.g., using random access addressing, or using direct memory access DMA, etc.) by devices such as data processor 1007.

Communications link 1015 can be configured to transmit (e.g., send, receive, signal, etc.) any types of communications packets (e.g., communication packet 1038₁, communication packet 1038_{N}) comprising any organization of data items. The data items can comprise a payload data area 1037, a destination address 1036 (e.g., a destination IP address), a source address 1035 (e.g., a source IP address), and can include various encodings or formatting of bit fields to populate packet characteristics 1034. In some cases, the packet characteristics include a version identifier, a packet or payload length, a traffic class, a flow label, etc. In some cases, payload data area 1037 comprises a data structure that is encoded and/or formatted to fit into byte or word boundaries of the packet.

In some embodiments, hard-wired circuitry may be used in place of or in combination with software instructions to implement aspects of the disclosure. Thus, embodiments of the disclosure are not limited to any specific combination of hardware circuitry and/or software. In embodiments, the term “logic” shall mean any combination of software or hardware that is used to implement all or part of the disclosure.

The term “computer readable medium” or “computer usable medium” as used herein refers to any medium that participates in providing instructions to data processor 1007 for execution. Such a medium may take many forms including, but not limited to, non-volatile media and volatile media. Non-volatile media includes, for example, optical or magnetic disks such as disk drives or tape drives. Volatile media includes dynamic memory such as RAM.

Common forms of computer readable media include, for example, floppy disk, flexible disk, hard disk, magnetic tape, or any other magnetic medium; CD-ROM or any other optical medium; punch cards, paper tape, or any other physical medium with patterns of holes; RAM, PROM, EPROM, FLASH-EPROM, or any other memory chip or cartridge, or any other non-transitory computer readable medium. Such data can be stored, for example, in any form of external data repository 1031, which in turn can be formatted into any one or more storage areas, and which can comprise parameterized storage 1039 accessible by a key (e.g., filename, table name, block address, offset address, etc.).

Execution of the sequences of instructions to practice certain embodiments of the disclosure are performed by a single instance of a computer system 10A00. According to certain embodiments of the disclosure, two or more instances of computer system 10A00 coupled by a communications link 1015 (e.g., LAN, public switched telephone network, or wireless network) may perform the sequence of instructions required to practice embodiments of the disclosure using two or more instances of components of computer system 10A00.

Computer system 10A00 may transmit and receive messages such as data and/or instructions organized into a data structure (e.g., communications packets). The data structure can include program instructions (e.g., application code 1003), communicated through communications link 1015 and communications interface 1014. Received program instructions may be executed by data processor 1007 as it is received and/or stored in the shown storage device or in or upon any other non-volatile storage for later execution. Computer system 10A00 may communicate through a data interface 1033 to a database 1032 on an external data repository 1031. Data items in a database can be accessed using a primary key (e.g., a relational database primary key).

Processing element partition 1001 is merely one sample partition. Other partitions can include multiple data processors, and/or multiple communications interfaces, and/or multiple storage devices, etc. within a partition. For example, a partition can bound a multi-core processor (e.g., possibly including embedded or co-located memory), or a partition can bound a computing cluster having plurality of computing elements, any of which computing elements are connected directly or indirectly to a communications link. A first partition can be configured to communicate to a second partition. A particular first partition and particular second partition can be congruent (e.g., in a processing element array) or can be different (e.g., comprising disjoint sets of components).

A module as used herein can be implemented using any mix of any portions of the system memory and any extent of hard-wired circuitry including hard-wired circuitry embodied as a data processor 1007. Some embodiments include one or more special-purpose hardware components (e.g., power control, logic, sensors, transducers, etc.). Some embodiments of a module include instructions that are stored in a memory for execution so as to facilitate operational and/or performance characteristics pertaining to processing deniable digital health diagnoses. A module may include one or more state machines and/or combinational logic used to implement or facilitate the operational and/or performance characteristics pertaining to processing deniable digital health diagnoses.

Various implementations of database 1032 comprise storage media organized to hold a series of records or files such that individual records or files are accessed using a name or key (e.g., a primary key or a combination of keys and/or query clauses). Such files or records can be organized into one or more data structures (e.g., data structures used to implement or facilitate aspects of deniable digital health diagnoses). Such files, records, or data structures can be brought into and/or stored in volatile or non-volatile memory. More specifically, the occurrence and organization of the foregoing files, records, and data structures improve the way that the computer stores and retrieves data in memory, for example, to improve the way data is accessed when the computer is performing operations pertaining to forming and handling deniable digital health diagnoses, and/or for improving the way data is manipulated when performing computerized operations pertaining to implementing an anonymous health data management framework.

FIG. 10B depicts an environment 10B00 in which embodiments of the present disclosure can operate. As an option, one or more aspects shown in environment 10B00 or any combination of components of the environment may be implemented in the context of the architecture and functionality of the embodiments described herein.

As shown, environment 10B00 comprises various computing systems (e.g., servers and devices) interconnected by a network 1050. The network 1050 can comprise any combination of a wide area network (e.g., WAN), local area network (e.g., LAN), cellular network, wireless LAN (e.g., WLAN), or any such means for enabling communication of computing systems. Some or all of network 1050 can also be referred to as “the Internet” or as an “Internet”. The example environment 10B00 comprises data collection devices 1060, an instance of a data management server 1062, an instance of a content storage facility 1063, and optional instances of third-party services 1064, all of which may communicate with any other operational elements over network 1050.

The servers and devices shown in environment 10B00 can represent any single computing system with dedicated hardware and software, or the servers and devices shown in environment 10B00 can represent multiple computing systems connected together (e.g., in a server farm, or in a host farm, etc.). In some cases, multiple computing systems share resources. For example, data management server 1062 and content storage facility 1063 might be closely coupled (e.g., co-located) and/or might be implemented using the same hardware platform.

The environment 10B00 may further comprise a variety of other devices such as a mobile phone 1051, a laptop 1052, a desktop computer 1053, a tablet 1054, a web camera 1055, a wearable device 1056, etc. The environment may still further comprise computing equipment such as a router 1057, an imaging device 1058 (e.g., CT scanner, MRI machine, etc.), and any number of storage devices 1059, etc. Some or all of the foregoing computing devices and computing equipment may support software (e.g., a browser, mobile application, etc.) and hardware (e.g., an LCD display, a graphics processing unit, display, monitor, etc.) capable of processing and displaying information (e.g., an image, a web page, etc.). Any of the foregoing computing devices or computing equipment can serve as or augment the capabilities of one of the data collection devices 1060.

In some embodiments, any particular one of the data collection devices 1060 can be used in conjunction with a different particular one of the data collection devices to determine the location and/or identity of a user.

As shown, the computing devices and computing equipment can perform a set of high-level interactions (e.g., operations, messages, etc.) in a protocol 1070. Specifically, the protocol can represent interactions in systems that facilitate deniable digital health diagnoses.

An application or app can be generated using any known techniques. Such an application or app cooperates with other operational elements of the environment to perform operations that facilitate deniable digital health diagnoses. The application or app may be configured so as to operate on any one or more data collection devices. As shown, any of the data collection devices 1060 can download such an application or app (operation 1082) from data management server 1062 or another other server, check the download for integrity, and then install the application or app (operation 1083). The application can be used to capture and/or generate data (operation 1084), process the captured or generated data (operation 1085₁), and submit data (message 1086) to the data management server.

To perform one or more operations of protocol 1070, data management server 1062 is configured to receive data (operation 1088) corresponding to the data submitted from the data collection devices. Such received data may be relayed or otherwise transmitted (message 1089₁ or message 1089₂) to downstream computing equipment such as the shown one or more third-party services 1064. The third-party services can process such data (e.g., operation 1085₂), possibly in response to the specific contents of the relayed or otherwise transmitted messages.

Furthermore, data management server 1062 may retrieve data (message 1090₁) from any storage facility, including from content storage facility 1063 or from any one or more of the third-party services (message 1090₂).

An instance of data management server 1062 can be configured to autonomously (e.g., under program control) analyze or otherwise process any received data (operation 1085₃). Moreover, example instances of data management server 1062 can be configured to store data at any storage facility, including at content storage facility 1063 (message 1096) or at any one or more storage devices of the third-party services 1064.

In some cases, the third-party services produce additional data that is derived, directly or indirectly, from the data received from the data collection devices. In some cases, and as shown, such additional data might be retrieved (message 1090₂) and analyzed or otherwise processed by data management server 1062 (operation 1085₃). As such, data can be transformed in a cascading fashion. Specifically, data can be initially processed at one or more of the data collection devices, then alternatively or additionally, the resulting data can be processed at the data management server, then alternatively or additionally, the still further resulting data can be processed at the third-party services. Furthermore, in some cases, data can be exchanged between content storage facility 1063 and any of the data collection devices 1060 (exchange 1098₁). Additionally, or alternatively, data can be exchanged between content storage facility 1063 and any of the third-party services 1064 (exchange 1098₂).

In the foregoing specification, the disclosure has been described with reference to specific embodiments thereof. It will however be evident that various modifications and changes may be made thereto without departing from the broader spirit and scope of the disclosure. For example, the above-described process flows are described with reference to a particular ordering of process actions. However, the ordering of many of the described process actions may be changed without affecting the scope or operation of the disclosure. The specification and drawings are to be regarded in an illustrative sense rather than in a restrictive sense.

What is claimed is:

1. A biomaterial delivery kit used in delivering deniable digital health diagnoses to an Internet-accessible location, the biomaterial delivery kit comprising:
a user card, the user card having printed thereon an identification code; and
a biomaterial container configured to have the same identification code as the identification code of the user card and configured to contain subject biomaterials that correspond to a subject anonymous user;
wherein anonymous health data that corresponds to anonymous analysis results of the subject biomaterials are published to a data repository; and
wherein access requests issued to the data repository are processed to match the subject anonymous user with one or more entries of the data repository; and
wherein the identification code of the user card is used to access at least one of the one or more entries that correspond to the subject anonymous user.

2. The biomaterial delivery kit of claim 1, wherein the identification code of the user card serves to prevent access to the one or more entries by users other than the subject anonymous user.

3. The biomaterial delivery kit of claim 1, wherein at least some of the entries are encrypted prior to being published to the data repository.

4. The biomaterial delivery kit of claim 1, wherein the subject anonymous user is matched with the at least one of the entries, based at least in part on a successful decryption of the at least one of the entries.

5. The biomaterial delivery kit of claim 1, wherein the entries are published to one or more partitions of the data repository with an association to one or more container barcode attributes.

6. The biomaterial delivery kit of claim 5, wherein the one or more container barcode attributes comprise at least one of, an encryption keyword, or a publication channel.

7. The biomaterial delivery kit of claim 5, wherein
one or more digest entries corresponding to the subject biomaterials are published to the data repository.

8. The biomaterial delivery kit of claim 7, wherein the digest entries are encrypted prior to being published to the data repository.

9. The biomaterial delivery kit of claim 7, wherein the access requests are processed to match the subject anonymous user with at least one of the digest entries of the data repository in response to analysis of the subject biomaterials from the subject anonymous user.

10. The biomaterial delivery kit of claim 9, wherein the subject anonymous user is matched with the at least one of the one or more digest entries, based at least in part on a successful decryption of the at least one of the digest entries.

11.-30. (canceled)

31. The biomaterial delivery kit of claim 1, wherein the biomaterial container and the user card both have the identification code and wherein the identification code does not include any personally identifiable information of the subject anonymous user.

32. The biomaterial delivery kit of claim 1, wherein the identification code is represented as machine-readable symbols or as a machine-readable barcode.

33. The biomaterial delivery kit of claim 32, wherein the user card comprises a set of card barcode attributes.

34. The biomaterial delivery kit of claim 33, wherein at least one of the card barcode attributes describes a user data request key.

35. The biomaterial delivery kit of claim 33, wherein at least one of the card barcode attributes comprises an encryption keyword.

36. The biomaterial delivery kit of claim 35, wherein the encryption keyword is used to determine if a decryption attempt is successful.

37. The biomaterial delivery kit of claim 35, wherein at least one of the card barcode attributes describes a publication channel.

38. The biomaterial delivery kit of claim 37, wherein the encryption keyword is also the publication channel.

39. The biomaterial delivery kit of claim 37, wherein the publication channel attribute is used to determine a target partition of the data repository.

40. The biomaterial delivery kit of claim 33, wherein the biomaterial container comprises set of container barcode attributes.

41. The biomaterial delivery kit of claim 40 wherein at least one of the container barcode attributes describes a publication service endpoint.

42. The biomaterial delivery kit of claim 40, wherein at least one of the container barcode attributes describes a user data publication key.

43. The biomaterial delivery kit of claim 1, further comprising access to executable code that is posted as a downloadable instance of a software application.

44. The biomaterial delivery kit of claim 43, further comprising executable code installed on a user device, the executable code to invoke one or more of the access requests to an anonymous data repository.

45. The biomaterial delivery kit of claim 44, wherein at least one of the access requests to the anonymous data repository is invoked by scanning a barcode on the user card.

46. The biomaterial delivery kit of claim 45, wherein at least one of the access requests comprises a user data request key derived from the barcode.

47. The biomaterial delivery kit of claim 44, wherein, in response to a successful decryption of at least one of the entries, the executable code publishes an anonymous response to an anonymous response repository.

48. The biomaterial delivery kit of claim 47, wherein the anonymous response is encrypted using a user data publication key.

49. The biomaterial delivery kit of claim 47, wherein the anonymous response comprises a question.

50. The biomaterial delivery kit of claim 47, wherein the anonymous response repository is accessible by at least one healthcare provider at the Internet-accessible location.
